SEQUENCE LISTING (9 sequences)

SEQ ID NO: 1 - protein, 510 aa, Simian immunodeficiency virus
  misc_feature (489)..(489): Xaa can be any naturally occurring amino acid
MGARNSVLSGKKADELEKIRLRPNGKKKYMLKHVVWAANELDRFGLAESLLENKEGCQKILSVLAPLVPTGSENLKSLYNTVCVIWCIHAEEKVKHTEEAKQIVQRHLVVETGTTETMPKTSRPTAPSSGRGGNYPVQQIGGNYVHLPLSPRTLNAWVKLIEEKKFGAEVVPGFQALSEGCTPYDINQMLNCVGDHQAAMQIIRDIINEEAADWDLQHPQPAPQQGQLREPSGSDIAGTTSSVDEQIQWMYRQQNPIPVGNIYRRWIQLGLQKCVRMYNPTNILDVKQGPKEPFQSYVDRFYKSLRAEQTDAAVKNWMTQTLLIQNANPDCKLVLKGLGVNPTLEEMLTACQGVGGPGQKARLMAEALKEALAPVPIPFAAAQQRGPRKTIKCWNCGKEGHSARQCRAPRRQGCWKCGKMDHVMAKCPDRQAGFLGLGPWGKKPRNFPMAQVHQGLMPTAPPEDPAVDLLKNYMQLGKQQREKQRESRXKPYKEVTEDLLHLNSLFGGDQ

SEQ ID NO: 2 - protein, 307 aa, Simian immunodeficiency virus
DQGSAINFGRGLAESLLENKEGCQKILSVLAPLVPTGSENLKSLYNTVCVIWCIHAEEKVKHTEEAKQIVQRHLVVETGTTETMPKTSRPTAPSSGRGGNYPVQQIGGNYVHLPLSPRTLNAWVKLIEEKKFGAEVVPGFQALSEGCTPYDINQMLNCVGDHQAAMQIIRDIINEEAADWDLQHPQPAPQQGQLREPSGSDIAGTTSSVDEQIQWMYRQQNPIPVGNIYRRWIQLKESSIGTSLGKAVHQVFGSVYTTMFGGVSWMIRILIGFLVLWIGTNSRNTSMAMTCIAVGGITLFLGFTVGA

SEQ ID NO: 3 - protein, 10 aa, Yellow fever virus
DQGSAINFGR

SEQ ID NO: 4 - protein, 6 aa, Yellow fever virus
KESSIG

SEQ ID NO: 5 - protein, 66 aa, Dengue virus type 4
TSLGKAVHQVFGSVYTTMFGGVSWMIRILIGFLVLWIGTNSRNTSMAMTCIAVGGITLFLGFTVGA

SEQ ID NO: 6 - DNA, 924 bp, Simian immunodeficiency virus
gatcaaggaagcgccatcaacttcggccgaggactggctgaatcactgctggaaaacaaagaaggatgtcagaaaatcctgtcagtgctggctccactggtgccaacaggatcagaaaacctgaagtcactgtacaacacagtgtgtgtgatctggtgtatccatgctgaagaaaaagtgaagcatacagaagaggccaagcagatcgtgcagaggcatctggtggtggaaacagggacaacagaaacaatgccaaagacatcaaggccaacagctccatcatcaggaagggggggaaactacccagtgcagcagatcggagggaactacgtgcatctgccactgtcaccaaggacactgaacgcttgggtgaaactgatcgaagaaaagaaatttggagctgaagtggtgccaggatttcaggctctgtcagaaggatgtacaccatacgacatcaaccagatgctgaactgtgtgggagatcatcaggctgctatgcagatcattagggacatcatcaacgaagaagctgcagattgggatctgcaacatccacagccagctccacagcagggacagctgagggaaccatcaggatcagacattgctggaacaacatcatcagtggacgaacagatccagtggatgtacaggcagcagaacccaatcccagtgggaaacatctacagaagatggatccagctgaaagagggaagctcaataggaacatcattgggaaaggctgtgcaccaggtttttggaagtgtgtatacaaccatgtttggaggagtctcatggatgattagaatcctaattgggttcttagtgttgtggattggcacgaactccaggaacacttcaatggctatgacgtgcatagctgttggaggaatcactctgtttctgggcttcacagttggcgcc

SEQ ID NO: 7 - DNA, 11785 bp, Simian immunodeficiency virus
agtaaatcctgtgtgctaattgaggtgcattggtctgcaaatcgagttgctaggcaataaacacatttggattaattttaatcgttcgttgagcgattagcagagaactgaccagaacatgtctggtcgtaaagctcagggaaaaaccctgggcgtcaatatggtacgacgaggagttcgctccttgtcaaacaaaataaaacaaaaaacaaaacaaattggaaacagacctggaccttcaagaggtgttcaaggatttatctttttctttttgttcaacattttgactggaaaaaagatcacagcccacctaaagaggttgtggaaaatgctggacccaagacaaggcttggctgttctaaggaaagtcaagagagtggtggccagtttgatgagaggattgtcctcaaggaaacgccgttcccatgatgttctgactgtgcaattcctaattttgggaatgctgttgatgaccggtggagtgaccttggtgcggaaaaacagatggttgctcctaaatgtgacatctgaggacctcgggaaaacattctctgtgggcacaggcaactgcacaacaaacattttggaagccaagtactggtgcccagactcaatggaatacaactgtcccaatctcagtccaagagaggagccagatgacattgattgctggtgctatggggtggaaaacgttagagtcgcatatggtaagtgtgactcagcaggcaggtctaggaggtcaagaagggccattgacttgcctacgcatgaaaaccatggtttgaagacccggcaagaaaaatggatgactggaagaatgggtgaaaggcaactccaaaagattgagagatggttcgtgaggaaccccttttttgcagtgacggctctgaccattgcctaccttgtgggaagcaacatgacgcaacgagtcgtgattgccctactggtcttggctgttggtccggcctactcagctcactgcattggaattactgacagggatttcattgagggggtgcatggaggaacttgggtttcagctaccctggagcaagacaagtgtgtcactgttatggcccctgacaagccttcattggacatctcactagagacagtagccattgatagacctgctgaggcgaggaaagtgtgttacaatgcagttctcactcatgtgaagattaatgacaagtgccccagcactggagaggcccacctagctgaagagaacgaaggggacaatgcgtgcaagcgcacttattctgatagaggctggggcaatggctgtggcctatttgggaaagggagcattgtggcatgcgccaaattcacttgtgccaaatccatgagtttgtttgaggttgatcagaccaaaattcagtatgtcatcagagcacaattgcatgtaggggccaagcaggaaaattggaataccagcattaagactctcaagtttgatgccctgtcaggctcccaggaagtcgagttcattgggtatggaaaagctacactggaatgccaggtgcaaactgcggtggactttggtaacagttacatcgctgagatggaaacagagagctggatagtggacagacagtgggcccaggacttgaccctgccatggcagagtggaagtggcggggtgtggagagagatgcatcatcttgtcgaatttgaacctccgcatgccgccactatcagagtactggccctgggaaaccaggaaggctccttgaaaacagctcttactggcgcaatgagggttacaaaggacacaaatgacaacaacctttacaaactacatggtggacatgtttcttgcagagtgaaattgtcagctttgacactcaaggggacatcctacaaaatatgcactgacaaaatgttttttgtcaagaacccaactgacactggccatggcactgttgtgatgcaggtgaaagtgtcaaaaggaaccccctgcaggattccagtgatagtagctgatgatcttacagcggcaatcaataaaggcattttggttacagttaaccccatcgcctcaaccaatgatgatgaagtgctgattgaggtgaacccaccttttggagacagctacattatcgttgggagaggagattcacgtctcacttaccagtggcacaaagagggaagctcaataggaaagttgttcactcagaccatgaaaggcgtggaacgcctggccgtcatgggagacaccgcctgggatttcagctccgctggagggttcttcacttcggttgggaaaggaattcatacggtgtttggctctgcctttcaggggctatttggcggcttgaactggataacaaaggtcatcatgggggcggtactcatatgggttggcatcaacacaagaaacatgacaatgtccatgagcatgatcttggtaggagtgatcatgatgtttttgtctctaggagttggggcggatcaaggaagcgccatcaacttcggccgaggactggctgaatcactgctggaaaacaaagaaggatgtcagaaaatcctgtcagtgctggctccactggtgccaacaggatcagaaaacctgaagtcactgtacaacacagtgtgtgtgatctggtgtatccatgctgaagaaaaagtgaagcatacagaagaggccaagcagatcgtgcagaggcatctggtggtggaaacagggacaacagaaacaatgccaaagacatcaaggccaacagctccatcatcaggaagggggggaaactacccagtgcagcagatcggagggaactacgtgcatctgccactgtcaccaaggacactgaacgcttgggtgaaactgatcgaagaaaagaaatttggagctgaagtggtgccaggatttcaggctctgtcagaaggatgtacaccatacgacatcaaccagatgctgaactgtgtgggagatcatcaggctgctatgcagatcattagggacatcatcaacgaagaagctgcagattgggatctgcaacatccacagccagctccacagcagggacagctgagggaaccatcaggatcagacattgctggaacaacatcatcagtggacgaacagatccagtggatgtacaggcagcagaacccaatcccagtgggaaacatctacagaagatggatccagctgaaagagggaagctcaataggaacatcattgggaaaggctgtgcaccaggtttttggaagtgtgtatacaaccatgtttggaggagtctcatggatgattagaatcctaattgggttcttagtgttgtggattggcacgaactccaggaacacttcaatggctatgacgtgcatagctgttggaggaatcactctgtttctgggcttcacagttggcgccgatcaaggatgcgccatcaactttggcaagagagagctcaagtgcggagatggtatcttcatatttagagactctgatgactggctgaacaagtactcatactatccagaagatcctgtgaagcttgcatcaatagtgaaagcctctttcgaagaagggaagtgtggcctaaattcagttgactcccttgagcatgagatgtggagaagcagggcagatgagattaataccatttttgaggaaaacgaggtggacatttctgttgtcgtgcaggatccaaagaatgtttaccagagaggaactcatccattttccagaattcgggatggtctgcagtatggttggaagacttggggtaagaaccttgtgttctccccagggaggaagaatggaagcttcatcatagatggaaagtccaggaaagaatgcccgttttcaaaccgggtctggaattctttccagatagaggagtttgggacgggagtgttcaccacacgcgtgtacatggacgcagtctttgaatacaccatagactgcgatggatctatcttgggtgcagcggtgaacggaaaaaagagtgcccatggctctccaacattttggatgggaagtcatgaagtaaatgggacatggatgatccacaccttggaggcattagattacaaggagtgtgagtggccactgacacatacgattggaacatcagttgaagagagtgaaatgttcatgccgagatcaatcggaggcccagttagctctcacaatcatatccctggatacaaggttcagacgaacggaccttggatgcaggtaccactagaagtgaagagagaagcttgcccagggactagcgtgatcattgatggcaactgtgatggacggggaaaatcaaccagatccaccacggatagcgggaaagttattcctgaatggtgttgccgctcctgcacaatgccgcctgtgagcttccatggtagtgatgggtgttggtatcccatggaaattaggccaaggaaaacgcatgaaagccatctggtgcgctcctgggttacagctggagaaatacatgctgtcccttttggtttggtgagcatgatgatagcaatggaagtggtcctaaggaaaagacagggaccaaagcaaatgttggttggaggagtagtgctcttgggagcaatgctggtcgggcaagtaactctccttgatttgctgaaactcacagtggctgtgggattgcatttccatgagatgaacaatggaggagacgccatgtatatggcgttgattgctgccttttcaatcagaccagggctgctcatcggctttgggctcaggaccctatggagccctcgggaacgccttgtgctgaccctaggagcagccatggtggagattgccttgggtggcgtgatgggcggcctgtggaagtatctaaatgcagtttctctctgcatcctgacaataaatgctgttgcttctaggaaagcatcaaataccatcttgcccctcatggctctgttgacacctgtcactatggctgaggtgagacttgccgcaatgttcttttgtgccatggttatcataggggtccttcaccagaatttcaaggacacctccatgcagaagactatacctctggtggccctcacactcacatcttacctgggcttgacacaaccttttttgggcctgtgtgcatttctggcaacccgcatatttgggcgaaggagtatcccagtgaatgaggcactcgcagcagctggtctagtgggagtgctggcaggactggcttttcaggagatggagaacttccttggtccgattgcagttggaggactcctgatgatgctggttagcgtggctgggagggtggatgggctagagctcaagaagcttggtgaagtttcatgggaagaggaggcggagatcagcgggagttccgcccgctatgatgtggcactcagtgaacaaggggagttcaagctgctttctgaagagaaagtgccatgggaccaggttgtgatgacctcgctggccttggttggggctgccctccatccatttgctcttctgctggtccttgctgggtggctgtttcatgtcaggggagctaggagaagtggggatgtcttgtgggatattcccactcctaagatcatcgaggaatgtgaacatctggaggatgggatttatggcatattccagtcaaccttcttgggggcctcccagcgaggagtgggagtggcacagggaggggtgttccacacaatgtggcatgtcacaagaggagctttccttgtcaggaatggcaagaagttgattccatcttgggcttcagtaaaggaagaccttgtcgcctatggtggctcatggaagttggaaggcagatgggatggagaggaagaggtccagttgatcgcggctgttccaggaaagaacgtggtcaacgtccagacaaaaccgagcttgttcaaagtgaggaatgggggagaaatcggggctgtcgctcttgactatccgagtggcacttcaggatctcctattgttaacaggaacggagaggtgattgggctgtacggcaatggcatccttgtcggtgacaactccttcgtgtccgccatatcccagactgaggtgaaggaagaaggaaaggaggagctccaagagatcccgacaatgctaaagaaaggaatgacaactgtccttgattttcatcctggagctgggaagacaagacgtttcctcccacagatcttggccgagtgcgcacggagacgcttgcgcactcttgtgttggcccccaccagggttgttctttctgaaatgaaggaggcttttcacggcctggacgtgaaattccacacacaggctttttccgctcacggcagcgggagagaagtcattgatgccatgtgccatgccaccctaacttacaggatgttggaaccaactagggttgttaactgggaagtgatcattatggatgaagcccattttttggatccagctagcatagccgctagaggttgggcagcgcacagagctagggcaaatgaaagtgcaacaatcttgatgacagccacaccgcctgggactagtgatgaatttccacattcaaatggtgaaatagaagatgttcaaacggacatacccagtgagccctggaacacagggcatgactggatcctggctgacaaaaggcccacggcatggttccttccatccatcagagctgcaaatgtcatggctgcctctttgcgtaaggctggaaagagtgtggtggtcctgaacaggaaaacctttgagagagaataccccacgataaagcagaagaaacctgactttatattggccactgacatagctgaaatgggagccaacctttgcgtggagcgagtgctggattgcaggacggcttttaagcctgtgcttgtggatgaagggaggaaggtggcaataaaagggccacttcgtatctccgcatcctctgctgctcaaaggagggggcgcattgggagaaatcccaacagagatggagactcatactactattctgagcctacaagtgaaaataatgcccaccacgtctgctggttggaggcctcaatgctcttggacaacatggaggtgaggggtggaatggtcgccccactctatggcgttgaaggaactaaaacaccagtttcccctggtgaaatgagactgagggatgaccagaggaaagtcttcagagaactagtgaggaattgtgacctgcccgtttggctttcgtggcaagtggccaaggctggtttgaagacgaatgatcgtaagtggtgttttgaaggccctgaggaacatgagatcttgaatgacagcggtgaaacagtgaagtgcagggctcctggaggagcaaagaagcctctgcgcccaaggtggtgtgatgaaagggtgtcatctgaccagagtgcgctgtctgaatttattaagtttgctgaaggtaggaggggagctgctgaagtgctagttgtgctgagtgaactccctgatttcctggctaaaaaaggtggagaggcaatggataccatcagtgtgttcctccactctgaggaaggctctagggcttaccgcaatgcactatcaatgatgcctgaggcaatgacaatagtcatgctgtttatactggctggactactgacatcgggaatggtcatctttttcatgtctcccaaaggcatcagtagaatgtctatggcgatgggcacaatggccggctgtggatatctcatgttccttggaggcgtcaaacccactcacatctcctatgtcatgctcatattctttgtcctgatggtggttgtgatccccgagccagggcaacaaaggtccatccaagacaaccaagtggcatacctcattattggcatcctgacgctggtttcagcggtggcagccaacgagctaggcatgctggagaaaaccaaagaggacctctttgggaagaagaacttaattccatctagtgcttcaccctggagttggccggatcttgacctgaagccaggagctgcctggacagtgtacgttggcattgttacaatgctctctccaatgttgcaccactggatcaaagtcgaatatggcaacctgtctctgtctggaatagcccagtcagcctcagtcctttctttcatggacaaggggataccattcatgaagatgaatatctcggtcataatgctgctggtcagtggctggaattcaataacagtgatgcctctgctctgtggcatagggtgcgccatgctccactggtctctcattttacctggaatcaaagcgcagcagtcaaagcttgcacagagaagggtgttccatggcgttgccaagaaccctgtggttgatgggaatccaacagttgacattgaggaagctcctgaaatgcctgccctttatgagaagaaactggctctatatctccttcttgctctcagcctagcttctgttgccatgtgcagaacgcccttttcattggctgaaggcattgtcctagcatcagctgccttagggccgctcatagagggaaacaccagccttctttggaatggacccatggctgtctccatgacaggagtcatgagggggaatcactatgcttttgtgggagtcatgtacaatctatggaagatgaaaactggacgccgggggagcgcgaatggaaaaactttgggtgaagtctggaagagggaactgaatctgttggacaagcgacagtttgagttgtataaaaggaccgacattgtggaggtggatcgtgatacggcacgcaggcatttggccgaagggaaggtggacaccggggtggcggtctccagggggaccgcaaagttaaggtggttccatgagcgtggctatgtcaagctggaaggtagggtgattgacctggggtgtggccgcggaggctggtgttactacgctgctgcgcaaaaggaagtgagtggggtcaaaggatttactcttggaagagacggccatgagaaacccatgaatgtgcaaagtctgggatggaacatcatcaccttcaaggacaaaactgatatccaccgcctagaaccagtgaaatgtgacacccttttgtgtgacattggagagtcatcatcgtcatcggtcacagagggggaaaggaccgtgagagttcttgatactgtagaaaaatggctggcttgtggggttgacaacttctgtgtgaaggtgttagctccatacatgccagatgttcttgagaaactggaattgctccaaaggaggtttggcggaacagtgatcaggaaccctctctccaggaattccactcatgaaatgtactacgtgtctggagcccgcagcaatgtcacatttactgtgaaccaaacatcccgcctcctgatgaggagaatgaggcgtccaactggaaaagtgaccctggaggctgacgtcatcctcccaattgggacacgcagtgttgagacagacaagggacccctggacaaagaggccatagaagaaagggttgagaggataaaatctgagtacatgacctcttggttttatgacaatgacaacccctacaggacctggcactactgtggctcctatgtcacaaaaacctcaggaagtgcggcgagcatggtaaatggtgttattaaaattctgacatatccatgggacaggatagaggaggtcaccagaatggcaatgactgacacaaccccttttggacagcaaagagtgtttaaagaaaaagttgacaccagagcaaaggatccaccagcgggaactaggaagatcatgaaagttgtcaacaggtggctgttccgccacctggccagagaaaagagccccagactgtgcacaaaggaagaatttattgcaaaagtccgaagtcatgcagccattggagcttacctggaagaacaagaacagtggaagactgccaatgaggctgtccaagacccaaagttctgggaactggtggatgaagaaaggaagctgcaccaacaaggcaggtgtcggacttgtgtgtacaacatgatggggaaaagagagaagaagctgtcagagtttgggaaagcaaagggaagccgtgccatatggtatatgtggctgggagcgcggtatcttgagtttgaggccctgggattcctgaatgaggaccattgggcttccagggaaaactcaggaggaggagtggaaggcattggcttacaatacctaggatatgtgatcagagacctggctgcaatggatggtggtggattctacgcggatgacaccgctggatgggacacgcgcatcacagaggcagaccttgatgatgaacaggagatcttgaactacatgagcccacatcacaaaaaactggcacaagcagtgatggaaatgacatacaagaacaaagtggtgaaagtgttgagaccagccccaggagggaaagcctacatggatgtcataagtcgacgagaccagagaggatccgggcaggtagtgacttatgctctgaacaccatcaccaacttgaaagtccaattgatcagaatggcagaagcagagatggtgatacatcaccaacatgttcaagattgtgatgaatcagttctgaccaggctggaggcatggctcactgagcacggatgtaacagactgaagaggatggcggtgagtggagacgactgtgtggtccggcccatcgatgacaggttcggcctggccctgtcccatctcaacgccatgtccaaggttagaaaggacatatctgaatggcagccatcaaaagggtggaatgattgggagaatgtgcccttctgttcccaccacttccatgaactacagctgaaggatggcaggaggattgtggtgccttgccgagaacaggacgagctcattgggagaggaagggtgtctccaggaaacggctggatgatcaaggaaacagcttgcctcagcaaagcctatgccaacatgtggtcactgatgtattttcacaaaagggacatgaggctactgtcattggctgtttcctcagctgttcccacctcatgggttccacaaggacgcacaacatggtcgattcatgggaaaggggagtggatgaccacggaagacatgcttgaggtgtggaacagagtatggataaccaacaacccacacatgcaggacaagacaatggtgaaaaaatggagagatgtcccttatctaaccaagagacaagacaagctgtgcggatcactgattggaatgaccaatagggccacctgggcctcccacatccatttagtcatccatcgtatccgaacgctgattggacaggagaaatacactgactacctaacagtcatggacaggtattctgtggatgctgacctgcaactgggtgagcttatctgaaacaccatctaacaggaataaccgggatacaaaccacgggtggagaaccggactccccacaacctgaaaccgggatataaaccacggctggagaaccgggctccgcacttaaaatgaaacagaaaccgggataaaaactacggatggagaaccggactccacacattgagacagaagaagttgtcagcccagaaccccacacgagttttgccactgctaagctgtgaggcagtgcaggctgggacagccgacctccaggttgcgaaaaacctggtttctgggacctcccaccccagagtaaaaagaacggagcctccgctaccaccctcccacgtggtggtagaaagacggggtctagaggttagaggagaccctccagggaacaaatagtgggaccatattgacgccagggaaagaccggagtggttctctgcttttcctccagaggtctgtgagcacagtttgctcaagaataagcagacctttggatgacaaacacaaaaccac

SEQ ID NO: 8 - protein, 3718 aa, Simian immunodeficiency virus
SGRKAQGKTLGVNMVRRGVRSLSNKIKQKTKQIGNRPGPSRGVQGFIFFFLFNILTGKKITAHLKRLWKMLDPRQGLAVLRKVKRVVASLMRGLSSRKRRSHDVLTVQFLILGMLLMTGGVTLVRKNRWLLLNVTSEDLGKTFSVGTGNCTTNILEAKYWCPDSMEYNCPNLSPREEPDDIDCWCYGVENVRVAYGKCDSAGRSRRSRRAIDLPTHENHGLKTRQEKWMTGRMGERQLQKIERWFVRNPFFAVTALTIAYLVGSNMTQRVVIALLVLAVGPAYSAHCIGITDRDFIEGVHGGTWVSATLEQDKCVTVMAPDKPSLDISLETVAIDRPAEARKVCYNAVLTHVKINDKCPSTGEAHLAEENEGDNACKRTYSDRGWGNGCGLFGKGSIVACAKFTCAKSMSLFEVDQTKIQYVIRAQLHVGAKQENWNTSIKTLKFDALSGSQEVEFIGYGKATLECQVQTAVDFGNSYIAEMETESWIVDRQWAQDLTLPWQSGSGGVWREMHHLVEFEPPHAATIRVLALGNQEGSLKTALTGAMRVTKDTNDNNLYKLHGGHVSCRVKLSALTLKGTSYKICTDKMFFVKNPTDTGHGTVVMQVKVSKGTPCRIPVIVADDLTAAINKGILVTVNPIASTNDDEVLIEVNPPFGDSYIIVGRGDSRLTYQWHKEGSSIGKLFTQTMKGVERLAVMGDTAWDFSSAGGFFTSVGKGIHTVFGSAFQGLFGGLNWITKVIMGAVLIWVGINTRNMTMSMSMILVGVIMMFLSLGVGADQGSAINFGRGLAESLLENKEGCQKILSVLAPLVPTGSENLKSLYNTVCVIWCIHAEEKVKHTEEAKQIVQRHLVVETGTTETMPKTSRPTAPSSGRGGNYPVQQIGGNYVHLPLSPRTLNAWVKLIEEKKFGAEVVPGFQALSEGCTPYDINQMLNCVGDHQAAMQIIRDIINEEAADWDLQHPQPAPQQGQLREPSGSDIAGTTSSVDEQIQWMYRQQNPIPVGNIYRRWIQLKEGSSIGTSLGKAVHQVFGSVYTTMFGGVSWMIRILIGFLVLWIGTNSRNTSMAMTCIAVGGITLFLGFTVGADQGCAINFGKRELKCGDGIFIFRDSDDWLNKYSYYPEDPVKLASIVKASFEEGKCGLNSVDSLEHEMWRSRADEINTIFEENEVDISVVVQDPKNVYQRGTHPFSRIRDGLQYGWKTWGKNLVFSPGRKNGSFIIDGKSRKECPFSNRVWNSFQIEEFGTGVFTTRVYMDAVFEYTIDCDGSILGAAVNGKKSAHGSPTFWMGSHEVNGTWMIHTLEALDYKECEWPLTHTIGTSVEESEMFMPRSIGGPVSSHNHIPGYKVQTNGPWMQVPLEVKREACPGTSVIIDGNCDGRGKSTRSTTDSGKVIPEWCCRSCTMPPVSFHGSDGCWYPMEIRPRKTHESHLVRSWVTAGEIHAVPFGLVSMMIAMEVVLRKRQGPKQMLVGGVVLLGAMLVGQVTLLDLLKLTVAVGLHFHEMNNGGDAMYMALIAAFSIRPGLLIGFGLRTLWSPRERLVLTLGAAMVEIALGGVMGGLWKYLNAVSLCILTINAVASRKASNTILPLMALLTPVTMAEVRLAAMFFCAMVIIGVLHQNFKDTSMQKTIPLVALTLTSYLGLTQPFLGLCAFLATRIFGRRSIPVNEALAAAGLVGVLAGLAFQEMENFLGPIAVGGLLMMLVSVAGRVDGLELKKLGEVSWEEEAEISGSSARYDVALSEQGEFKLLSEEKVPWDQVVMTSLALVGAALHPFALLLVLAGWLFHVRGARRSGDVLWDIPTPKIIEECEHLEDGIYGIFQSTFLGASQRGVGVAQGGVFHTMWHVTRGAFLVRNGKKLIPSWASVKEDLVAYGGSWKLEGRWDGEEEVQLIAAVPGKNVVNVQTKPSLFKVRNGGEIGAVALDYPSGTSGSPIVNRNGEVIGLYGNGILVGDNSFVSAISQTEVKEEGKEELQEIPTMLKKGMTTVLDFHPGAGKTRRFLPQILAECARRRLRTLVLAPTRVVLSEMKEAFHGLDVKFHTQAFSAHGSGREVIDAMCHATLTYRMLEPTRVVNWEVIIMDEAHFLDPASIAARGWAAHRARANESATILMTATPPGTSDEFPHSNGEIEDVQTDIPSEPWNTGHDWILADKRPTAWFLPSIRAANVMAASLRKAGKSVVVLNRKTFEREYPTIKQKKPDFILATDIAEMGANLCVERVLDCRTAFKPVLVDEGRKVAIKGPLRISASSAAQRRGRIGRNPNRDGDSYYYSEPTSENNAHHVCWLEASMLLDNMEVRGGMVAPLYGVEGTKTPVSPGEMRLRDDQRKVFRELVRNCDLPVWLSWQVAKAGLKTNDRKWCFEGPEEHEILNDSGETVKCRAPGGAKKPLRPRWCDERVSSDQSALSEFIKFAEGRRGAAEVLVVLSELPDFLAKKGGEAMDTISVFLHSEEGSRAYRNALSMMPEAMTIVMLFILAGLLTSGMVIFFMSPKGISRMSMAMGTMAGCGYLMFLGGVKPTHISYVMLIFFVLMVVVIPEPGQQRSIQDNQVAYLIIGILTLVSAVAANELGMLEKTKEDLFGKKNLIPSSASPWSWPDLDLKPGAAWTVYVGIVTMLSPMLHHWIKVEYGNLSLSGIAQSASVLSFMDKGIPFMKMNISVIMLLVSGWNSITVMPLLCGIGCAMLHWSLILPGIKAQQSKLAQRRVFHGVAKNPVVDGNPTVDIEEAPEMPALYEKKLALYLLLALSLASVAMCRTPFSLAEGIVLASAALGPLIEGNTSLLWNGPMAVSMTGVMRGNHYAFVGVMYNLWKMKTGRRGSANGKTLGEVWKRELNLLDKRQFELYKRTDIVEVDRDTARRHLAEGKVDTGVAVSRGTAKLRWFHERGYVKLEGRVIDLGCGRGGWCYYAAAQKEVSGVKGFTLGRDGHEKPMNVQSLGWNIITFKDKTDIHRLEPVKCDTLLCDIGESSSSSVTEGERTVRVLDTVEKWLACGVDNFCVKVLAPYMPDVLEKLELLQRRFGGTVIRNPLSRNSTHEMYYVSGARSNVTFTVNQTSRLLMRRMRRPTGKVTLEADVILPIGTRSVETDKGPLDKEAIEERVERIKSEYMTSWFYDNDNPYRTWHYCGSYVTKTSGSAASMVNGVIKILTYPWDRIEEVTRMAMTDTTPFGQQRVFKEKVDTRAKDPPAGTRKIMKVVNRWLFRHLAREKSPRLCTKEEFIAKVRSHAAIGAYLEEQEQWKTANEAVQDPKFWELVDEERKLHQQGRCRTCVYNMMGKREKKLSEFGKAKGSRAIWYMWLGARYLEFEALGFLNEDHWASRENSGGGVEGIGLQYLGYVIRDLAAMDGGGFYADDTAGWDTRITEADLDDEQEILNYMSPHHKKLAQAVMEMTYKNKVVKVLRPAPGGKAYMDVISRRDQRGSGQVVTYALNTITNLKVQLIRMAEAEMVIHHQHVQDCDESVLTRLEAWLTEHGCNRLKRMAVSGDDCVVRPIDDRFGLALSHLNAMSKVRKDISEWQPSKGWNDWENVPFCSHHFHELQLKDGRRIVVPCREQDELIGRGRVSPGNGWMIKETACLSKAYANMWSLMYFHKRDMRLLSLAVSSAVPTSWVPQGRTTWSIHGKGEWMTTEDMLEVWNRVWITNNPHMQDKTMVKKWRDVPYLTKRQDKLCGSLIGMTNRATWASHIHLVIHRIRTLIGQEKYTDYLTVMDRYSVDADLQLGELI

SEQ ID NO: 9 - protein, 508 aa, Human immunodeficiency virus
MGARASVLSGGKLDKWEKIRLRPGGKKTYQLKHIVWASRELERFAVNPGLLETGGGCKQILVQLQPSLQTGSEELKSLYNAVATLYCVHQGIEVRDTKEALDKIEEEQNKSKKKAQQAAADTGNSSQVSQNYPIVQNLQGQMVHQAISPRTLNAWVKVIEEKAFSPEVIPMFSALSEGATPQDLNTMLNTVGGHQAAMQMLKETINEEAAEWDRLHPAHAGPNAPGQMREPRGSDIAGTTSTLQEQIGWMTSNPPVPVGEIYKRWIILGLNKIVRMYSPVSILDIRQGPKEPFRDYVDRFYKTLRAEQASQDVKNWMTETLLVQNANPDCKTILKALGPAATLEEMMTACQGVGGPSHKARILAEAMSQVTSPANIMMQRGNFRNQRKTIKCFNCGKEGHLARHCRAPRKKGCWKCGREGHQMKDCTERQANFLGKIWPSHKGRPGNFLQSRPEPTAPPEESFRFGEETTTPPQKQEPLPSQKQETIDKDLYPLASLKSLFGNDPSLQ

(19) United States
(12) Patent Application Publication
(10) Pub. No.: US 2012/0321655 A1
(43) Pub. Date: Dec. 20, 2012

(54) LENTIVIRUS VACCINE BASED ON THE RECOMBINANT VIRAL VACCINE AGAINST YELLOW FEVER

(76) Inventors: Myrna Cristina Bonaldo, Jacarepagua (BR); Ricardo Galler, Pendotiba (BR); David Ian Watkins, Arena, WI (US); Jonah Bradley Sacha, Madison, WI (US)

(21) Appl. No.: 13/518,876

(22) PCT Filed: Dec. 22, 2010
(86) PCT No.: PCT/BR10/00430

§ 371 Date: Sep. 6, 2012

(30) Foreign Application Priority Data
Dec. 23, 2009 (BR) PI0914507-9

(51) Int. Cl. A61K 39/21; C12P 21/02; A61P 31/18; C07K 14/155; C12N 15/49

(52) U.S. Cl. 424/188.1; 530/350; 536/23.72; 424/199.1; 435/69.3

(57) ABSTRACT

The present invention relates to an attenuated, recombinant viral vaccine against yellow fever which expresses heterologous sequences of a lentivirus and is used as an immunisation agent to induce an immune response to lentivirus.

Part of the described material has been developed with the financial support of the National Institutes of Health of the United States of America under number [identification code] R11 AI076114. Thus, the North American government may have certain rights concerning the described technical material.

### FIELD OF THE INVENTION

The present invention relates to the development of a vaccine against lentivirus, and more specifically against primate lentiviruses such as HIV (Human Immunodeficiency Virus) and SIV (Simian Immunodeficiency Virus).

The present invention concerns the use of attenuated recombinant yellow fever vaccine virus 17D for expression of HIV and SIV antigens for immunization. The invention provides immunization schemes using yellow fever virus expressing HIV or SIV antigens. For example, the recombinant virus may be used in a “prime-boost” protocol immunizing an individual with a recombinant polynucleotide or recombinant Bacillus Calmette-Guérin (rBCG) vaccine, which expresses one or more HIV or SIV antigens, followed by booster dose with recombinant Yellow Fever virus 17D, which expresses one or more HIV or SIV antigens.

### BACKGROUND OF THE ART

Over the 28-year period since the scientists identified the human immunodeficiency virus (HIV) as the cause of the acquired immunodeficiency syndrome (AIDS), the virus has spread inexorably, giving rise to one of the most devastating pandemics ever recorded in history. More than 20 million people have already died of AIDS and about 33 million people are living with HIV infection.

However, none of the vaccine regimens tested in HIV vaccine efficacy trials to date has reduced HIV infection or replication rates.

Structural aspects and the enormous variability of the Envelope glycoprotein have frustrated the efforts to broadly induce reactive neutralizing antibodies against HIV [1]. Investigators have therefore focused their attention on T-cell-based vaccines. The recent trial of a recombinant adenovirus[Ad5]-vectored vaccine was widely seen as an important test of this concept [3,4]. Unfortunately, vaccinees became infected at higher rates than control groups [3].

Several studies have shown the key role of CD+8 T cells in the control of HIV infections as well as of simian immunodeficiency virus (SIV) infections. As a result, various modalities of response-inducing vaccines regarding CD8+ T-cell effector response have been currently investigated and developed [5, 6, 7, 8].

Hence, it is also desirable that new approaches are developed in order to reduce the incidence of HIV infection or improve the consequences of the infection. As regards this aspect, vaccines are particularly relevant.

### SUMMARY OF THE INVENTION

The technical matter described herein relates to pharmaceutical compositions that may be used as immunogenic compounds or vaccines against lentivirus, and more specifically as immunogenic compounds or vaccines against HIV (Human Immunodeficiency Virus) or SIV (Simian Immunodeficiency Virus).

### BRIEF DESCRIPTION OF THE FIGURES

FIG. 1 shows viral growth curves in Vero cells.

FIG. 2 shows that YF17D replicates and induces neutralizing antibodies, virus-specific CD8+ T cells, and CD8+ T-cell activation in rhesus monkeys.

FIG. 3 shows the study of infection and immunogenicity of YF17D/SIVGag45-269 virus in rhesus monkeys.

FIG. 4 shows that vaccination with rYF17D/SIVGag45-269 yielded a robust expansion of Gag-specific responses in a monkey initially vaccinated with rBCG.

FIG. 5 shows that the result of the vaccination with rYF17D/SIVGag45-269 of r01056 [sic].

### DETAILED DESCRIPTION OF THE INVENTION

The described compositions typically include a recombinant polynucleotide or a panel of recombinant polynucleotides. Adequate HIV or SIV polynucleotides may encode at least one fragment of a HIV or SIV polypeptide including Gag, Pol, Rev, Tat, Nef, Vif, Vpx, Vpr, Env, Vpu, [sic] (preferably at least one fragment of Gag, Vif, and Nef). The recombinant polynucleotide or panel of recombinant polynucleotides may be present in a recombinant virus or in a panel of recombinant viruses which express polypeptides encoded by the recombinant polynucleotides. The disclosed compositions may be used in methods for inducing immune response against HIV, which may include HIV-1 or HIV-2, or SIV (for example, response based on cytotoxic T cells (CTC), and, optionally, humoral or antibody-based response. [sic]

Disclosed compositions may include a mixture of recombinant polynucleotides, which may be present in a viral vector or in other nucleotide vector such as a plasmid or a bifunctional vector (“shuttle” vectors). Typically, the polynucleotides are present in expression vectors to express the polypeptides encoded by the polynucleotides. Adequate vectors may include but are not limited to viral vectors (for example, attenuated recombinant viruses). The pharmaceutical composition may include free recombinant nucleic acid or recombinant nucleic acid packaged into one or more virus particles (for example, replication-defective viral particles or attenuated virus). For instance, the recombinant polynucleotides contemplated herein may be present in one or more recombinant viruses (for example, recombinant yellow fever viruses) and the compositions may include a mixture of recombinant viruses.

The compositions may include a polynucleotide or a panel of polynucleotides (for example, at least about 2-9 polynucleotides) present in one or more expression vectors (for example, a viral vector or other expression vector, [sic] which encode the complete Gag polypeptide or a fragment/part of it. Optionally, the compositions may include a polynucleotide or a panel of polynucleotides present in one or more expression vectors which encode the complete Vif polypeptide or a fragment/part of it. Optionally, the compositions may include a polynucleotide or a panel of polynucleotides present in one or more expression vectors which encode the complete Nef polypeptide or a fragment/part of it. Optionally, the compositions may include a polynucleotide or a panel of polynucleotides present in one or more expression vectors which encode the complete Tat polypeptide or a fragment/part of it. Optionally, the compositions may include a polynucleotide or a panel of polynucleotides present in one or more expression vectors which encode Rev polypeptide or a fragment/part of it. Optionally, the compositions may include a polynucleotide or a panel of polynucleotides present in one or more expression vectors which encode the complete Pol polypeptide or a fragment/part of it. Optionally, the compositions may include a polynucleotide or a panel of polynucleotides present in one or more expression vectors which encode the complete Vpr polypeptide or a fragment/part of it. Optionally, the compositions may include a polynucleotide or a panel of polynucleotides present in one or more expression vectors which encode the complete Vpx polypeptide or a fragment/part of it. The polynucleotides or panels of polynucleotides may encode different polypeptides or different fragments of polypeptides. The encoded polypeptides or fragments of polypeptides may overlap (for example, in about 9-20 amino acids). The fragments are typically in the length range of 5-100 amino acids (or from 5-50, 10-50, 10-25, 5-15, or 10-15 amino acids) and include at least one epitope of the polypeptide from which the fragment is derived. In some embodiments, a fragment may include an N-terminal amino acid which is not normally present in the complete polypeptide from which the fragment is derived.

The pharmaceutical composition may comprise an effective amount or concentration of recombinant polynucleotides or recombinant viruses to induce a protective or therapeutic immune response against HIV infection in humans or SIV infection in simians. Inducing a protective or therapeutic immune response may include the potentiation of a CD8+ and/or CD4+ response to one or more HIV or SIV epitopes. Inducing a protective response may include the induction of sterilizing immunity against HIV or SIV. Inducing a therapeutic response may include the reduction of the viral load of an individual, for example, as determined by the measurement of the amount of circulating virus before and after delivering the composition. In some embodiments, the viral load is reduced to less than about 10,000 vRNA copies/mL (preferably to less than about 5,000 vRNA copies/mL, more preferably to less than about 2000 vRNA copies/mL, much more preferably to less than about 1500 vRNA copies/mL, and even more preferably to less than about 1000 vRNA copies/mL) at least about 3 months after the composition administration. The induction of a therapeutic response may include the increase in CD4+ T-cell count of the individual (for example, at least a twofold, threefold, or fourfold increase) at least 3 months after the composition administration.

Methods inducing protective or therapeutic immune responses against HIV or SIV infection through the administration of the pharmaceutical compositions described herein (for example, as immunogenic compounds or vaccines) to an individual who needs them, which may include a human who has already acquired or is at risk of acquiring a lentivirus infection, are also disclosed.

As used herein, a “nucleic acid vaccine” or “free DNA vaccine” relates to a vaccine which includes one or more expression vectors which encode B-cell and/or T-cell epitopes and provide an immunoprotective response to the individual who is being vaccinated. Nucleic acid vaccines as defined here, which may include expression vector plasmids, are not typically encapsidated into a viral particle. The vaccine nucleic acid is directly introduced into the cells of the individual who is subjected to the vaccinal regimen. This approach is described, for example, in U.S. Pat. Nos. 5,580,859; 5,589,466; 5,804,566; 5,739,118; 5,736,524; 5,679,647; and WO 98/04720. Examples of DNA-based administration technologies may include pure DNA (“naked DNA”) administration, facilitated distribution (for example, through the use of bupivacaine, polymers, or peptides), and administration in the presence of cationic lipid complex or liposomes. Nucleic acids may be delivered using ballistic administration (for example, as described in U.S. Pat. No. 5,204,253), by pressure (for example, as described in U.S. Pat. No. 5,922,687), or by electroporation.

The nucleic acids used in the compositions and vaccines disclosed herein may encode one or more viral proteins or portions of one or more viral proteins. In some embodiments, the nucleic acids may encode an epitope (for example, a T-cell epitope) which is not part of a known HIV or SIV functional protein or which is encoded by a HIV or SIV alternative open reading frame (for example, a “cryptic” epitope). (See, for example, Maness et al., J. Exp. Med. (2007), 204:2505-12; and Cardinaud et al., J. Exp. Med. (2004), 199:1053-1063, which are all incorporated by reference herein in their entireties). A cryptic epitope may be located in an open reading frame (ORF) whose translation is not typically observed in the course of HIV or SIV viral replication. For example, Maness et al. discloses a cryptic epitope in SIVmac239 called “cRW9”, which is located in the +2 reading frame relative to the ORF encoding the envelope protein. This cryptic epitope is located in the same ORF that encodes exon 1 of the Rev protein, but it is downstream of the only known splice donor site and so is not predicted to be translated under “normal” biological circumstances.

The pharmaceutical compositions disclosed herein may be formulated as vaccines for administration to an individual who needs them. These compositions may be formulated and/or administered at dosages and through techniques that are well known by physicians, taking into account factors such as age, gender, weight, particular conditions of the patient, and route of administration. The compositions may include pharmaceutical carriers, diluents, or excipients as they are known [in the art]. Moreover, the compositions may include preservatives (for example, antimicrobial or antibacterial agents such as benzalkonium chloride) or adjuvants.

The pharmaceutical compositions may be administered prophylactically or therapeutically. In prophylactic administration, vaccines may be administered in a sufficient amount to induce CD8+, CD4+, and/or antibody responses for protection against infection. In therapeutic applications, vaccines are administered to a patient in a sufficient amount to induce a therapeutic effect (for example, CD8+, CD4+, and/or antibody responses for HIV or SIV antigens or epitopes encoded by the polynucleotides of the composition [sic], which heals or at least partially suspends or delays the symptoms and/or complications of HIV or SIV infection (i.e., a “therapeutically effective dose”).

The compositions disclosed herein may include recombinant polynucleotides or recombinant viruses which encode and express complete HIV or SIV polypeptides or their fragments. As used herein, a “minigene” encodes only part of a gene. For example, a minigene for HIV Gag may encode only 5-100 amino acids of the Gag polypeptide (or 5-50, 10-50, 5-25, 10-25, 5-15, or 10-15 amino acids). Minigenes which encode only part of a gene are described in the U.S. patent application Ser. No. 12/022,530, filed on Jan. 30, 2008, which claims the benefit of U.S. Provisional Application No. 60/898,644, filed on Jan. 31, 2007, the contents of which are incorporated by reference herein in their entireties. For example, a minigene for HIV Gag may encode a Gag fragment which includes only 5-100 amino acids of the Gag polypeptide (or 5-50, 10-50, 5-25, 10-25, 5-15, or 10-15 amino acids). In some embodiments, the recombinant polynucleotide or recombinant viruses may encode and express a SIV polypeptide fragment as disclosed in Table 1 or a corresponding HIV polypeptide fragment.

TABLE 1
Epitope-specific sequences of amino acids.

| | | | MHC class I |
|---|---|---|---|
| Protein | Name | Sequence | restriction |
| Tat | SL8 | STPESANL | A*01 |
| Nef | IW9 | IRYPKTFGW | B*17 |
| | MW9 | MHPAQTSQW | B*17 |
| | YY9 | YTSGPGIRY | A*02 |
| | YY9* | YTYEAYVRY | A*02 |
| | AL11 | ARRHRILDIYL | B*08 |
| | ML10 | MRRSRPSGDL | B*08 |
| | RL10 | RRRLTARGLL | B*08 |
| Vif | HW8 | HLEVQGYW | B*17 |
| | WY8 | WTDVTPNY | A*02 |
| | RL8 | RRDNRRGL | B*08 |
| | RL9 | RRAIRGEQL | B*08 |
| Ver | KL9 | KRLRLIHLL | B*08 |
| | RL10 | RRRWQQLLAL | B*08 |
| Pol p10 | LV10 | LGPHYTPKIV | A*01 |
| protease | YL8 | YHSNVKEL | A*07 |
| Env gp120 | CL9 | CAPPGYALL | A*01 |
| Env gp41 | TL9 | TVPWPNASL | A*01 |
| Env gp41 | RY8 | RTLLSRVY | A*02 |
| Env gp41 | FW9 | FHEAVQAVW | B*17 |
| Gag p17 | GY9 | GSENLKSLY | A*02 |
| matrix | CM9 | CTPYDINQM | A*01 |
| Gag p27 | QI9 | QNPIPVGNI | A*01 |
| capsid | LF8 | LAPVPIPF | A*01 |
| Gag p27 | RW9 | RAPRRQGCW | B*17 |
| capsid | | | |
| Vpx | II11 | IPPGNSGEETI | A*01 |

In some embodiments, the recombinant polynucleotide or recombinant viruses may comprise a “minigene” of SIV or HIV and express a SIV polypeptide fragment or a corresponding HIV polypeptide fragment as disclosed in Tables 2-6.

TABLE 2
SIV and HIV Gag Protein Fragments

| Protein | Sequence |
|---|---|
| 1(a) | SVLSGKKADELEKIRLRPNGKKKYMLKHVVWAANELDRFGLAESLLE |
| SIV Gag p17 | |
| 1(b) | SVLSGGKLDKWEKIRLRPGGKKTYQLKHIVWASRELERFAVNPGLLE |
| HIV Gag p17 | |
| 2(a) | FGLAESLLENKEGCQKILSVLAPLVPTGSENLKSLYNTVDV |
| SIV Gag p17 | |
| 2(b) | FAVNPGLLETGGGCKQILVQLQPSLQTGSEELKSLYNAVAT |
| HIV Gag p17 | |
| 3(a) | NKEGCQKILSVLAPLVPTGSENLKSLYNTVCVIWCIHAEEKVKHTEEA |
| SIV Gag p17 | KQIVQRHLVVETGTTETMPKTSR |
| 3(b) | TGGGCKQILVQLQPSLQTGSEELKSLYNAVATLYCVHQGIEVRDTKEA |
| HIV Gag p17 | LDKIEEEQNKSKKKAQQAAA |
| 4(a) | GNYVHLPLSPRTLNAWVKLIEEKKFGAEVVPGFQALSEGCTPYDI |
| SIV Gag p27 | |
| 4(b) | GQMVHQAISPRTLNAWVKVIEEKAFSPEVIPMFSALSEGATPQDL |
| HIV Gag p24 | |
| 5(a) | SEGCTPYDINQMLNCVGDHQAAMQIIRDIINEEAADWDLQHPQPAPQQ |
| SIV Gag p27 | GQLREPSGSDIAGTTSSVDEQIQWMYRQQNPIP |
| 5(b) | SEGATPQDLNTMLNTVGGHQAAMQMLKETINEEAAEWDRLHPAHAGPN |
| HIV Gag p24 | APGQMREPRGSDIAGTTSTLQEQIGWMTSNPPVP |
| 6(a) | SEGCTPYDINQMLNCVGDHQAAMQIIRDIINEEAADWDLQHPQPAPQQ |
| SIV Gag p27 | GQ |
| 6(b) | SEGATPQDLNTMLNTVGGHQAAMQMLKETINEEAAEWDRLHPAHAGPN |
| HIV Gag p24 | APGQ |
| 7(a) | MYRQQNPIPVGNIYRRWIQLGLQKCVRMYNPTNIPDVKQGPKEPFQSY |
| SIV Gag p27 | VDRFYKSLRAEQTDAAVKNWMTQTLLIQNANPDCKLVLKGLGVNPTLE |
| | EMLTACQGVGGPGQKARLMAEALKEALAPVPIPFAAAQQRGPRKPIKC |
| | WNCGKEGHSARQCRAPRRQGCW |
| 7(b) | MTSNPPVPVGEIYKRWIILGLNKIVRMYSPVSILDIRQGPKEPFRDYV |
| HIV Gag p24 | DRFYKTLRAEQASQDVKNWMTETLLVQNANPDCKTILKALGPAATLEE |
| | MMTACQGVGGPSHKARILAEAMSQVTSPANIMMQRGNFRNQRKTIKCF |
| | NCGKEGHLARHCRAPRKKGCW |
| 8(a) | TTSSVDEQIQWMYRQQNPIPVGNIYRRWIQLGLQKCVRMYNPTNIPDV |
| SIV Gag p27 | KQGPKEPFQSYVDRFYKSLRAEQTDAAVKNWMTQTLLIQNANPDCKLV |
| | LKGLGVNPTLEEMLTACQGVGGPG |
| 8(b) | TTSTLQEQIGWMTSNPPVPVGEIYKRWIILGLNKIVRMYSPVSILDIR |
| HIV Gag p24 | QGPKEPFRDYVDRFYKTLRAEQASQDVKNWMTETLLVQNANPDCKTIL |
| | KALGPAATLEEMMTACQGVGGPS |

TABLE 3
SIV and HIV Vif Protein Fragments

| | |
|---|---|
| 10(a) | MEEEKRWIAVPTWRIPERLERWHSLIKYLKYKTKDLQKVCYVPHFKVGWA |
| SIV Vif | WWTCSRVIFPLQEGSHLEVQGYWHLTPEKGWLSTYAVRITWYSKNFWTDV |
| | TPNYADILLH |
| 10(b) | MENRWQVMIVWQVDRMRIKTWKSLVKHHMYISKKAKEWVYRHHYESTHPR |
| HIV Vif | ISSEVHIPLGDAKLVITTYWGLHTGEREWHLGQGVSIEWRKKRYNTQVDP |
| | DLADKLIH |
| 11(a) | PNYADILLHSTYFPCFTAGEVRRAIRGEQLLSCCRFPRAHKYQVPSLQYL |
| SIV Vif | ALKVVSDVRSQGENPTWKQWRRDNRRGLRMAKQNSRGDKQRGGKPPTKGA |
| | NFPGLAKVLGILA |
| 11(b) | PDLADKLIHLHYFDCFSDSAIRHAILGHRVRPKCEYQAGHNKVGSLQYLA |
| HIV Vif | LTALITPKKIKPPLPSVRKLTEDRWNKPQKTKGHRGSHTMNGH |
| 12(a) | MEEEKRWIAVPTWRIPERLERWHSLIKYLKYKTKDLQKVCYVPHFKVGWA |
| SIV Vif | WWTCSRVIFP |
| 12(b) | MENRWQVMIVWQVDRMRIKTWKSLVKHHMYISKKAKEWVYRHHYESTHPR |
| HIV Vif | ISSEVHIP |
| 13(a) | WWTCSRVIFPLQEGSHLEVQGYWHLTPEKGWLSTYAVRITWYSKNFWTDV |
| SIV Vif | TPNYADILLH |
| 13(b) | ESTHPRISSEVHIPLGDAKLVITTYWGLHTGEREWHLGQGVSIEWRKKRY |
| HIV Vif | NTQVDPDLADKLIH |
| 14(a) | PNYADILLHSTYFPCFTAGEVRRAIRGEQLLSCCRFPRAHKYQVPSLQYL |
| SIV Vif | ALKVVSDVR |
| 14(b) | PDLADKLIHLHYFDCFSDSAIRHAILGHRVRPKCEYQAGHNKVGSLQYLA |
| HIV Vif | LTALITPKK |
| 15(a) | LALKVVSDVRSQGENPTWKQWRRDNRRGLRMAKQNSRGDKQRGGKPPTKG |
| SIV Vif | ANFPGLAKVLGILA |
| 15(b) | LALTALITPKKIKPPLPSVRKLTEDRWNKPQKTKGHRGSHTMNGH |
| HIV Vif | |

TABLE 4
SIV and HIV Nef Protein Fragments

| | |
|---|---|
| 16(a) | GLDKGLSSLSCEGQKYNQGQYMNTPWRNPAEEREKLAYRKQNMDDIDEED |
| SIV Nef | DDLVGVSVRPKVPLRTMSYKLAIDMSHFIKEKGGLEGIYYSARRHRILDI |
| | YLEKEEGIIPDWQDYTSGPGIRYPKTFGWLWKLVPVNVSDEAQEDEEHYL |
| | MHPAQTSQWDDPWGEV |
| 16(b) | MGGKWSKSSKAGWPQVREKIKQTPPAAEGVGAVSQDLDKHGAITSSNMNN |
| HIV Nef | ADCVWLQAQEDEEVGFPVRPQVPLRPMTFKGAFDLSFFLKEKGGLDGLIY |
| | SRKRQEILDLWVYNTQGFFPDWQNYTPGPGVRLPLCFGWCFKLVPVDPRE |
| | VEEDNKGENNCLLHPLSQHGMEDEHKEV |
| 17(a) | GLDKGLSSLSCEGQKYNQGQYMNTPWRNPAEEREKLAYRKQNMDDIDEED |
| SIV Nef | DD |
| 17(b) | MGGKWSKSSKAGWPQVREKIKQTPPAAEGVGAVSQDLDKHGAITSSNMNN |
| HIV Nef | ADCVWLQAQEDEE |
| 18(a) | DDIDEEDDDLVGVSVRPKVPLRTMSYKLAIDMSHFIKEKGGLEGIYYSAR |
| SIV Nef | RH |
| 18(b) | DCVWLQAQEDEEVGFPVRPQVPLRPMTFKGAFDLSFFLKEKGGLDGLIYS |
| HIV Nef | RKR |
| 19(a) | GIYYSARRHRILDIYLEKEEGIIPDWQDYTSGPGIRYPKTFGWLWKLVPV |
| SIV Nef | |
| 19(b) | GLIYSRKRQEILDLWVYNTQGFFPDWQNYTPGPGVRLPLCFGWCFKLVPV |
| HIV Nef | |
| 20(a) | GWLWKLVPVNVSDEAQEDEEHYLMHPAQTSQWDDPWGEV |
| SIV Nef | |
| 20(b) | GWCFKLVPVDPREVEEDNKGENNCLLHPLSQHGMEDEHKEV |
| HIV Nef | |

TABLE 5
SIV and HIV Tat Protein Fragments

| | |
|---|---|
| 21(a) | METPLREQENSLESSNERSSCISEADASTPESANLGEEILSQLYRPLEA |
| SIV Tat | |
| 21(b) | MEPVDPRLEPWKHPGSQPKTPCTKCYCKKCCLHCQVCFMTKGLGISYGRK |
| HIV Tat | |
| 22(a) | SQLYRPLEACYNTCYCKKCCYHCQFCFLKKGLGICYEQSRKRRRTPKKAK |
| SIV Tat | |
| 22(b) | SQPKTPCTKCYCKKCCLHCQVCFMTKGLGISYGRKKRRQRRRAPQDNKNH |
| HIV Tat | |

TABLE 6
SIV and HIV Rev Protein Fragments

| | |
|---|---|
| 23(a) | MSNHEREEELRKRLRLIHLLHQTNPYPTGPGTANQRRQRKRRWRRRWQQ |
| SIV Rev | |
| 23(b) | MAGRSGSTDEELLRAVRIIKILYQSNPYPSSEGTRQARRNRRRRWRARQR |
| HIV Rev | |
| 24(a) | RRWRRRWQQLLALADRIYSFPDPPTDTPLDLAIQQLQNLAIESIPDPPTN |
| SIV Rev | TPEALCDPTEDSRSPQD |
| 24(b) | RRNRRRRWRARQREICALSERILSSCLGRPTEPVPLPLPPLERLTLDCSE |
| HIV Rev | DCGTSGTQQSQGTETGVGRPQISGE |

A minigene of SIV or a minigene of HIV may encode a polypeptide comprising a SIV or HIV polypeptide fragment and also comprise an N-terminal methionine. A minigene of SIV or a minigene of HIV may include codon sequences which are optimized for expression (for example, in animal, organism or recombinant virus as contemplated herein). Exemplary SIV minigenes including codon sequences which are optimized for expression by the yellow fever virus are depicted in Table 7.

TABLE 7
Optimized-Sequence Minigenes

| Name | |
|---|---|
| | 1. SIVmac239Gag(6-52) |
| DNA Seq. | 5′- |
| | GTCGACAGGAGCCACCATGAGCGTGCTGAGCGGCAAGAAGGCCGACGAGC |
| | TGGAGAAGATCCGGCTGCGGCCCAACGGCAAGAAGAAATACATGCTGAAG |
| | CACGTGGTGTGGGCCGCCAACGAGCTGGACCGGTTCGGCCTGGCCGAGAG |
| | CCTGCTGGAGTGAGAATTCGCGGCCGC-3′ |
| AA Seq. | MSVLSGKKADELEKIRLRPNGKKKYMLKHVVWAANELDRFGLAESLLE |
| | 2. SIVmac239Gag(44-84) |
| DNA Seq. | 5′- |
| | GTCGACAGGAGCCACCATGTTCGGCCTGGCCGAGAGCCTGCTGGAGAACA |
| | AGGAGGGCTGCCAGAAGATCCTGAGCGTGCTGGCCCCTCTGGTGCCCACC |
| | GGCAGCGAGAACCTGAAGAGCCTGTACAACACCGTGTGCGTGTGAGAATT |
| | CGCGGCCGC-3′ |
| AA Seq. | MFGLAESLLENKEGCQKILSVLAPLVPTGSENLKSLYNTVCV |
| | 3. SIVmac239Gag(76-123) |
| DNA Seq. | 5′- |
| | GTCGACAGGAGCCACCATGAAGAGCCTGTACAACACCGTGTGCGTGATCT |
| | GGTGCATCCACGCCGAGGAGAAGGTGAAGCACACCGAGGAGGCCAAGCAG |
| | ATCGTGCAGCGGCACCTGGTGGTGGAGACCGGCACCACCGAGACCATGCC |
| | CAAGACCAGCCGCTGAGAATTCGCGGCCGC-3′ |
| AA Seq. | MKSLYNTVCVIWCIHAEEKVKHTEEAKQIVQRHLVVETGTTETMPKTSR |
| | 4. SIVmac239Gag(142-186) |
| DNA Seq. | 5′- |
| | GTCGACAGGAGCCACCATGGGCAACTACGTGCACCTGCCCCTGAGCCCCC |
| | GGACCCTGAACGCCTGGGTGAAGCTGATCGAGGAGAAGAAGTTCGGCGCC |
| | GAGGTGGTGCCCGGCTTCCAAGCCCTGAGCGAGGGCTGCACCCCCTACGA |
| | CATCTGAGAATTCGCGGCCGC-3′ |
| AA Seq. | MGNYVHLPLSPRTLNAWVKLIEEKKFGAEVVPGFQALSEGCTPYDI |
| | 5. SIVmac239Gag(178-258) |
| DNA Seq. | 5′- |
| | GTCGACAGGAGCCACCATGAGCGAGGGCTGCACCCCCTACGACATCAACC |
| | AGATGCTGAACTGCGTGGGCGACCACCAGGCCGCCATGCAGATCATCCGG |
| | GACATCATCAACGAGGAGGCCGCCGACTGGGACCTGCAGCACCCCCAGCC |
| | CGCCCCCCAGCAAGGCCAGCTGCGGGAGCCCAGCGGCAGCGACATCGCCG |
| | GCACCACCAGCAGCGTGGACGAGCAGATCCAGTGGATGTACCGGCAGCAG |
| | AACCCCATCCCCTGAGAATTCGCGGCCGC-3′ |
| AA Seq. | MSEGCTPYDINQMLNCVGDHQAAMQIIRDIINEEAADWDLQHPQPAPQQG |
| | QLREPSGSDIAGTTSSVDEQIQWMYRQQNPIP |
| | 6. SIVmac239Gag(250-415) |
| DNA Seq. | 5′- |
| | GTCGACAGGAGCCACCATGTACCGGCAGCAGAACCCCATCCCCGTGGGCA |
| | ACATCTACCGGCGGTGGATCCAGCTGGGCCTGCAGAAATGCGTGCGGATG |
| | TACAACCCCACCAACATCCTGGACGTGAAGCAGGGCCCCAAGGAGCCCTT |
| | CCAGAGCTACGTGGACCGGTTCTACAAGAGCCTGCGGGCCGAGCAGACCG |
| | ACGCCGCCGTGAAGAACTGGATGACCCAGACCCTGCTGATCCAGAACGCC |
| | AACCCCGACTGCAAGCTGGTGCTGAAGGGCCTGGGCGTGAACCCCACCCT |
| | GGAGGAGATGCTGACCGCCTGCCAGGGCGTGGGCGGCCCCGGCCAGAAGG |
| | CCCGGCTGATGGCCGAGGCCCTGAAGGAGGCCCTGGCCCCCGTGCCCATC |
| | CCCTTCGCCGCCGCCCAGCAGCGGGGCCCCCGGAAGCCCATCAAGTGCTG |
| | GAACTGCGGCAAGGAGGGCCACAGCGCCCGGCAGTGCCGGGCCCCCCGGC |
| | GGCAGGGCTGCTGGTGAGAGAATTCGCGGCCGC-3′ |
| AA Seq. | MYRQQNPIPVGNIYRRWIQLGLQKCVRMYNPTNILDVKQGPKEPFQSYVD |
| | RFYKSLRAEQTDAAVKNWMTQTLLIQNANPDCKLVLKGLGVNPTLEEMLT |
| | ACQGVGGPGQKARLMAEALKEALAPVPIPFAAAQQRGPRKPIKCWNCGKE |
| | GHSARQCRAPRRQGCW |
| | 7. SIVmac239Vif(1-110) |
| DNA Seq. | 5′- |
| | GTCGACAGGAGCCACCATGGAGGAGGAGAAGCGGTGGATCGCCGTGCCCA |
| | CCTGGCGGATCCCCGAGCGGCTGGAGCGGTGGCACAGCCTGATCAAGTAC |
| | CTGAAGTACAAGACCAAGGACCTGCAGAAGGTGTGCTACGTGCCCCACTT |
| | CAAAGTGGGCTGGGCCTGGTGGACCTGCAGCCGGGTGATCTTCCCCCTGC |
| | AAGAGGGCAGCCACCTGGAGGTGCAGGGCTACTGGCACCTGACCCCCGAG |
| | AAGGGCTGGCTGAGCACCTACGCCGTGCGGATCACCTGGTACAGCAAGAA |
| | CTTCTGGACCGACGTGACCCCCAACTACGCCGACATCCTGCTGCACTGAG |
| | AATTCGCGGCCGC-3′ |
| AA Seq. | MEEEKRWIAVPTWRIPERLERWHSLIKYLKYKTKDLQKVCYVPHFKVGWA |
| | WWTCSRVIFPLQEGSHLEVQGYWHLTPEKGWLSTYAVRITWYSKNFWTDV |
| | TPNYADILLH |
| | 8. SIVmac239Vif(102-214) |
| DNA Seq. | 5′- |
| | GTCGACAGGAGCCACCATGCCCAACTACGCCGACATCCTGCTGCACAGCA |
| | CCTACTTCCCCTGCTTCACCGCCGGCGAGGTCCGGCGGGCCATCCGGGGC |
| | GAGCAGCTGCTGAGCTGCTGCCGGTTCCCCCGGGCCCACAAGTACCAGGT |
| | GCCCAGCCTGCAGTACCTGGCCCTGAAGGTGGTGAGCGACGTGCGGAGCC |
| | AGGGCGAGAACCCCACCTGGAAGCAGTGGCGGCGGGACAACCGGCGGGGC |
| | CTGCGGATGGCCAAGCAGAACAGCCGGGGCGACAAGCAGCGGGGCGGCAA |
| | GCCTCCCACCAAGGGCGCCAACTTCCCCGGCCTGGCCAAGGTGCTGGGCA |
| | TCCTGGCCTGAGAATTCGCGGCCGC-3′ |
| AA Seq. | MPNYADILLHSTYFPCFTAGEVRRAIRGEQLLSCCRFPRAHKYQVPSLQY |
| | LALKVVSDVRSQGENPTWKQWRRDNRRGLRMAKQNSRGDKQRGGKPPTKG |
| | ANFPGLAKVLGILA |
| | 9. SIVmac239Nef(45-210) |
| DNA Seq. | 5′- |
| | GTCGACAGGAGCCACCATGGGCCTGGACAAGGGCCTGAGCAGCCTGAGCT |
| | GCGAGGGCCAGAAGTACAACCAGGGCCAGTACATGAACACCCCCTGGCGG |
| | AACCCCGCCGAGGAGCGGGAGAAGCTGGCCTACCGGAAGCAGAACATGGA |
| | CGACATCGACGAGGAGGACGACGACCTGGTGGGCGTGAGCGTGCGGCCCA |
| | AGGTGCCCCTGCGGACCATGAGCTACAAGCTGGCCATCGACATGAGCCAC |
| | TTCATCAAGGAGAAGGGCGGCCTGGAGGGCATCTACTACAGCGCCCGGCG |
| | GCACCGGATCCTGGACATCTACCTGGAGAAGGAGGAGGGCATCATCCCCG |
| | ACTGGCAGGACTACACCAGCGGCCCCGGCATCCGGTACCCCAAGACCTTC |
| | GGCTGGCTGTGGAAGCTGGTGCCCGTGAACGTGAGCGACGAGGCCCAGGA |
| | GGACGAGGAGCACTACCTGATGCACCCCGCCCAGACCAGCCAGTGGGACG |
| | ACCCCTGGGGCGAGGTGTGAGAATTCGCGGCCGC-3′ |
| AA Seq. | MGLDKGLSSLSCEGQKYNQGQYMNTPWRNPAEEREKLAYRKQNMDDIDEE |
| | DDDLVGVSVRPKVPLRTMSYKLAIDMSHFIKEKGGLEGIYYSARRHRILD |
| | IYLEKEEGIIPDWQDYTSGPGIRYPKTFGWLWKLVPVNVSDEAQEDEEHY |
| | LMHPAQTSQWDDPWGEV |

The viral vectors, recombinant bacteria, and nucleic acids used in the compounds and vaccines disclosed herein may encode one or more proteins of HIV or SIV or portions of one or more proteins of HIV or SIV. In some embodiments, nucleic acids may encode an epitope (for example, a T-cell epitope) which is not part of a known HIV or SIV functional protein or which is encoded by a HIV or SIV alternative open reading frame (i.e., a “cryptic” epitope). (See, for example, Maness et al., J. Exp. Med. (2007), 204:2505-12; and Cardinau et al., J. Exp. Med. (2004), 199:1053-1063, which are all incorporated by reference herein in their entireties). A cryptic epitope may be located in an open reading frame (ORF) whose translation is not typically observed in the course of HIV or SIV viral replication. For example, Maness et al. discloses a cryptic epitope in SIVmac239 called “cRW9”, which is located in the +2 reading frame relative to the ORF encoding the envelope protein. This cryptic epitope is located in the same ORF that encodes exon 1 of the Rev protein, but it is at the 3′ side (“downstream”) of the donor processing site (“donor splicing site”) and so its translation would not be expected under “normal” biological conditions.

Any of the conventional vectors used for expression in eukaryotic cells may be used to directly immunize an individual with nucleic acid. Expression vectors containing regulatory elements of eukaryotic viruses may be used in eukaryotic expression vectors (for example, vectors containing SV40, CMV, or retroviral enhancers or promoters). Exemplary vectors include vectors expressing proteins under the direction of such promoters as SV40 early promoter, SV40 late promoter, metallothionein promoter, human cytomegalovirus promoter, murine mammary tumor virus promoter, and Rous sarcoma virus promoter. Industrial amounts for therapeutic and prophylactic use of plasmid DNA may be yielded, for example, by fermentation in E. coli, followed by purification. Aliquots from the working cell bank are used to inoculate a culture medium, and cultured until saturation in shake flasks or in a bioreactor according to widely known techniques. Plasmid DNA may be purified by standard bioseparation technologies such as solid phase ion exchange resins. If required, the DNA may be isolated from the open-circular and linear forms using gel electrophoresis or other methods.

The purified plasmid DNA may be prepared for injection using a wide range of formulations, such as, for example, lyophilized DNA which may be reconstituted in phosphate-buffered sterile saline (PBS) solution. The purified DNA may be introduced into an individual through any appropriate method; for example, by intramuscular (IM) or intradermal (ID) administration.

In order to maximize the immunotherapy effects of DNA vaccines, alternative methods may be desirable to formulate purified plasmid DNA. A variety of methods has been described, and new techniques may become available. For example, cationic lipids may be used in the formulation of the pharmaceutical composition disclosed herein (for example, as described in WO 93/24640; U.S. Pat. No. 5,279,833; and WO 91/06309). Protective, interactive, noncondensing compounds (PINC) may be used in the formulation of the pharmaceutical composition disclosed herein (for example, glycolipids, fusogenic liposomes, and peptides).

The term “vector” refers to a few means by which nucleic acid may be introduced into a host organism or tissue. There are several types of vectors, including viruses, plasmids, bacteriophages, cosmids, and bacteria. As used herein, a “recombinant virus” or “viral vector” relates to the recombinant viral nucleic acid which has been genetically modified to express a heterologous polypeptide (for example, a HIV polypeptide or a fragment thereof). The recombinant viral nucleic acid typically includes cis-acting elements for the expression of the heterologous polypeptide. Typically, the recombinant viral nucleic acid can be packaged into a virus which is able to infect a host cell. For example, the recombinant viral nucleic acid may include cis-acting elements for packaging. Generally, the viral vector is not able to replicate or is attenuated. An “attenuated recombinant virus” refers to a virus which has been genetically modified by modern molecular biological methods, making it less virulent than the wild type, usually by mutation or deletion of specific genes. For example, the recombinant viral nucleic acid may not have an essential gene for an efficient production or for the production of the infectious virus.

The recombinant viral nucleic acid may function as a vector for an immunogenic retroviral protein in view of the fact that the recombinant viral nucleic acid comprises exogenous nucleic acid. The recombinant virus may be introduced in a vaccinated human by standard methods for vaccination using live vaccines. A live vaccine as contemplated herein may be administered using, for example, from about 10⁴ to 10⁸ units of viral particles/dose, or 10⁶ to 10⁹ pfu/dose. The actual dosages of this vaccine may be readily determined by any expert in the vaccine field.

Numerous virus species may be used as recombinant virus vectors for the pharmaceutical composition disclosed herein. Preferred recombinant virus for a viral vaccine is the yellow fever vaccine virus (for example, 17-D or similar).

In some embodiments, attenuated recombinant bacteria or mycobacteria may be used as vectors in the pharmaceutical compositions and vaccines disclosed herein (for example, Bacillus, Shigella, Salmonella, Listeria or Yersinia, attenuated recombinant bacteria). Recombinant bacterial vaccine vectors are described by Daudel et al., “Use of attenuated bacteria as delivery vectors for DNA vaccines”, Expert Review of Vaccines, Volume 6, Number 1, February, 2007, pp. 97-110(14); Shata et al., “Recent advances with recombinant bacterial vaccine vectors”, Molec. Med. Today (2000), Volume 6, Number 2, Feb. 1, 2000, pages 66-71; Clare & Dougan, “Live Recombinant Bacterial Vaccines”, Novel Vaccination Strategies, Apr. 16, 2004 (Editor Stefan H. E. Kaufman); Gentaschev et al., “Recombinant Attenuated Bacteria for the Delivery of Subunit Vaccines”, Vaccine, Volume 19, Numbers 17-19, Mar. 21, 2001, pages 2621-2628; Garmory et al., “The Use of live attenuated bacteria as a delivery system for heterologous antigens”, J. Drug Target. 2003; 11 (8-10): 471-9; U.S. Pat. No. 6,383,496; and U.S. Pat. No. 6,923,958 (which are all incorporated by reference herein in their entireties).

In some embodiments, the recombinant bacterium or mycobacterium is the recombinant Bacillus Calmette-Guérin (or Bacillus Calmette-Guérin, BCG). The BCG is an attenuated strain of the live bovine tuberculosis bacillus, Mycobacterium bovis, which lost its virulence in humans by being specially cultured in an artificial medium over years. The use of a recombinant BCG (rBCG) expressing the codon-optimized HIV-1 Gag for mice immunization in a “prime-boost” regimen has been described. Promkhatkaaew D. et al., “Prime-boost immunization of codon optimized HIV-1 CRFO1 AE Gag in BCG with recombinant vaccinia virus elicits MHC class I and II immune responses in mice”, Immunol Invest. 2009; 38(8):762-79.

The compositions included in the vaccine regimen as contemplated herein may be coadministered or administered sequentially with another immunological antigenic vaccine or in therapeutic compositions, including an adjuvant, a biological or chemical agent given in combination with or genetically fused to an antigen to increase the antigen immunogenicity. Additional therapeutic agents may include but are not limited to CD40 or interleukin-2 (IL-2) ligand in a sufficient amount to further potentiate CD8+ T cell, CD4+ T cell, and antibody responses. These other compositions may also include purified antigens of the immunodeficiency virus or the expression of such antigens by a second recombinant vector system which can give rise to additional therapeutic compositions.

The pharmaceutical composition disclosed herein may be delivered by various routes. The typical delivery routes include parenteral administration (for example, intradermal, intramuscular, or subcutaneous delivery). Other routes include oral, intranasal, intravaginal, and intrarectal administration. The pharmaceutical composition may be formulated for intranasal or pulmonary delivery. The pharmaceutical composition formulations may include liquid formulations (for example, for oral, nasal, anal, or vaginal administration, among others, including suspensions, syrups, or elixirs) and preparations for parenteral, subcutaneous, intradermal, intramuscular, or intravenous administration (for example, injectable administration) such as sterile suspensions or emulsions.

As used herein, the term “adjuvant” refers to a compound or mixture which increases the immune response to an antigen. An adjuvant may serve as a tissue depot that slowly releases the antigen and also as a lymphoid system activator which nonspecifically amplifies the immune response. Examples of adjuvants which may be employed include the adjuvant MPL-TDM (Monophosphoryl lipid A/synthetic trehalose dicorynomycolate; for example, available from GSK Biologicals). Another appropriate adjuvant is the AS021/AS02 immunostimulatory adjuvant (GSK). These immunostimulatory adjuvants are formulated to promote an intense T-cell response and include QS-21, a saponin from Quillay saponaria, the TL4 ligand, a monophosphoryl lipid A, together in a lipid or liposomal carrier. Other adjuvants include but are not limited to nonionic block copolymer adjuvants (for example, CRL1005), aluminum phosphates (for example, A1PO4), R-848 (a Th1-like adjuvant), imiquimod, PAM3CYS, poly (I:C), loxoribine, potentially useful human adjuvants such as BCG (Bacillus Calmette-Guérin) and Corynebacterium parvum, CpG oligodeoxynucleotides (ODN), antigens derived from cholera toxin (for example, CTA1-DD), lipopolysaccharide adjuvants, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions in water (for example, MF59 available from Novartis Vaccines or Montanide ISA 720), keyhole-limpet hemocyanins, and dinitrophenol.

The pharmaceutical compositions disclosed herein may also or additionally comprise at least one antiviral chemotherapy compound. Nonlimiting examples may be selected from at least one compound of the group consisting of gamma globulin, amantadine, guanidine, benzimidazole hydroxide, interferons, interleukin-16, thiosemicarbazones, methisazones, rifampicin, ribavirin, pyrimidine analogs (for example, AZT and/or 3TC), purine analogs, foscarnet, phosphonoacetic acid, acyclovir, dideoxynucleosides, protease inhibitors (for example, saquinavir; indinavir; ritonavir; AG 1343; and VX-2/78), chemokines, such as RANTES, MIP1x or MIP1b, or ganciclovir.

As used herein, a “prime-boost vaccination regimen” refers to a regimen in which a first composition is administered to an individual one or more times (for example, two or three times with about 2, 3, or 4 weeks between administrations), and, after a certain period of time (for example, about two weeks, about four weeks, about two months, about three months, about four months, about five months, about six months, or more), a second composition is administered to the individual, which may be equal to or different from the first composition. The second composition may also be administered more than once, with at least 2, 3, or 4 weeks between administrations. In some embodiments, the individual is given the first composition, which comprises the recombinant yellow fever virus 17D expressing one or more HIV or SIV polypeptides, or their fragments, or rBCG expressing one or more HIV or SIV polypeptides, or their fragments. Subsequently, the individual receives the second composition, which comprises the recombinant yellow fever virus 17D expressing one or more HIV or SIV polypeptides, or their fragments, DNA encoding one or more HIV or SIV polypeptides, or their fragments, or rBCG expressing one or more HIV or SIV polypeptides or their fragments. The first and the second compositions may be the same or different.

The pharmaceutical compositions disclosed herein may be delivered to individuals at risk of developing HIV or SIV infection or to individuals who are already infected with HIV or SIV. To evaluate the efficacy of the vaccine, the immune response may be assessed by measuring the induction of CD8+ and antibody responses for particular epitopes. CD8+ T-cell responses may be measured, for example, by using cultured or fresh PBMC tetramer staining, ELISPOT assays, or functional cytotoxicity assays, which are well known among experts, and are described herein. Antibody responses may be measured by widely known assays such as ELISA. Determinations of viral titers or loads and CD4+ T cells after immunization may be performed in individuals who have already been infected. For example, individuals may present a decline in the viral load and an increase in the CD4+cell count after immunization (for example, at least a twofold, threefold, or fourfold increase in the CD4+cell count in relation to the count obtained prior to immunization).

The invention will now be described in detail regarding certain embodiments of the invention.

In some embodiments of the recombinant viruses contemplated herein, the virus is a yellow fever vaccinal virus engineered to express a heterologous polypeptide. The heterologous polypeptide may include a HIV polypeptide (HIV-1 or HIV-2), a SIV polypeptide, or a fragment of them (for example, a fragment consisting of at least 8, 9, 10, 20, 30, 40, 50, 100, or 200 contiguous amino acids of a HIV Gag polypeptide or a SIV Gag polypeptide). In other embodiments, the heterologous polypeptide is a polypeptide with at least 90%, 95%, 96%, 97%, 98%, or 99% of sequence identity with a HIV polypeptide, a SIV polypeptide, or a fragment of them (for example, a fragment consisting of at least 8, 9, 10, 20, 30, 40, 50, 100, or 200 contiguous amino acids of a HIV Gag polypeptide or a SIV Gag polypeptide). Sequences for HIV-1 strains and several genes of HIV-1 strains (including the gene for Gag polypeptide) are publicly available in GenBank; for example, the sequences available under accession numbers: NC_{—}001802; EU293448, EU293447, EU293446, EU293445, EU293444, FJ195091, FJ195090, FJ195089, FJ195088, FJ195087, FJ195086, EU884501, EU786681, EU786680, EU786679, EU786678, EU786677, EU786676, EU786675, EU786674, EU786673, EU786672, EU786671, EU786670, EU697909, EU697908, EU697907, EU697906, EU697905, EU697904, U71182, EU69324, EU861977, FJ213783, FJ213782, FJ213781, FJ213780, AB428562, AB428561, AB428560, AB428559, AB428558, AB428557, AB428556, AB428555, AB428554, AB428553, AB428552, AB428551, DQ295195, DQ295196, DQ295194, DQ295193, DQ295192, EU446022, EU735540, EU735539, EU735538, EU735537, EU735536, EU735535, EU110097, EU110096, EU110095, EU110094, EU110093, EU110092, EU110091, EU110090, EU110089, EU110088, EU110087, EU110086, EU110085, AF193277, AF193276, AF049337, EU541617, EU220698, EF469243, DQ912823, DQ912822, EU000516, EU000515, EU000514, EU000513, EU000512, EU000511, EU000510, EU000509, EU000508, EU000507, and EU884500; whose submissions to GenBank are incorporated by reference herein in their entireties. Primary and field isolates of HIV are available from the National Institute of Allergy and Infectious Diseases (NIAID), which has contracted the services of Quality Biological (Gaithersburg, Md.) to make these strains available. Strains are also available from the World Health Organization (WHO), Geneva, Switzerland. In a preferred embodiment, the selected polypeptide has a consensus sequence as determined by the comparison of three or more HIV strains. As disclosed herein, the polynucleotides encoding a HIV polypeptide or a SIV polypeptide may be “optimized” for expression in a human cellular environment or simian cellular environment, respectively. Thus, the use of polynucleotides specifically including HIV genes that are codon-optimized for expression in a human cellular environment is contemplated herein. Optimized synthetic HIV gag genes are described in U.S. Pat. No. 6,696,291.

The recombinant yellow fever viruses contemplated herein are prepared through the insertion of the sequence encoding the heterologous polypeptide into the genome of the yellow fever virus or vector which can replicate the yellow fever virus. Adequate yellow fever viruses include attenuated strains such as YF17D. Adequate insertion sites for the sequence encoding the heterologous polypeptide include sites between the sequences encoding viral proteins E and NS1. The sequence encoding the heterologous polypeptide may be codon-optimized based on the use of the codon for the yellow fever virus genome.

In another embodiment of the recombinant viruses described herein, the virus is a genetically modified vaccinal YF 17D virus which expresses amino acids 45-269 of SIVmac239Gag (rYF17D/SIVGag45-269), and is prepared through the insertion of a codon-optimized sequence based on YF genome between the genes which encode viral proteins E and NS1.

In one example, the antigen used for expression by the yellow fever 17D virus was a fragment of a Gag protein comprising amino acid residues 45 to 269. Gag is a precursory protein that, on being processed by the viral protease, generates the internal structural proteins of the mature virion. Gag protein induces a strong protective immune response mediated by CD8+ T cells in rhesus monkeys and, on a smaller scale, in humans. This immune response is very important since it may provide protection against viral infection or reduce the viral load and delay the onset of disease.

In some embodiments, the following criteria may be used to select a Gag fragment to be cloned and expressed in the E/NS1 region of 17D virus genome. First, the presence of CD8+ T-cell epitopes in the Gag polypeptide may be taken into consideration based on immunological studies in rhesus monkeys. Furthermore, preferably, the insertion of the Gag polypeptide should not disturb the translocation of the YF precursory protein into the endoplasmic reticulum, as well as its processing by viral and cell proteases, if these steps occur during virus morphogenesis and assembly. Thus, preferably, the Gag polypeptide fragment should not be hydrophobic or contain a transmembrane motif, which might produce a rupture or significantly destabilize the correct topology of the membrane and precursory protein processing. Therefore, the selected Gag polypeptide fragment is hydrophilic and does not include apparent transmembrane domains. Besides, a very flexible motif, KESSIG, is fused to the C-terminal of the Gag sequence. This sort of motif is present in all flaviviruses and proteins binding domain III and the anchorage region (“stem anchor”) of E protein. It is a very flexible motif and was used here as a separating sequence to connect and move away the Gag fragment and alpha helices of the anchorage domain (“stem anchor”) of truncated dengue 4 virus E protein in the recombinant cassette.

Moreover, the virus vaccines presently disclosed differ from the virus vaccines of the state of the art by using an attenuated vector structure and emphasizing the generation of cell immune responses for specific viral epitopes in certain proteins. The strategy for vector virus replication is maximizing the production of antigens in an appropriate cell location to induce an appropriate response in terms of specificity and magnitude.

Vaccination with a single dose of attenuated yellow fever virus 17D causes a limited viral infection and promotes the generation of neutralizing antibodies as well as T-cell responses which confer protective immunity to more than 95% of vaccinees, providing immunity for over 30 years. The yellow fever 17D vaccine is regarded as one of the most successful vaccines available. Vaccine production concerns a well-established process and, since 1937, over 540 million doses have been used in humans with a minimal incidence of severe side effects [9,10].

The current methods are based on the methodology that was previously developed by two of the present inventors. The methodology is described in WO07051267, which is incorporated by reference herein.

The method for the production of recombinant viruses with nucleotide sequences totally or partially encoding the heterologous proteins, as described in WO07051267, may comprise the following steps:

(a) modification of the heterologous nucleotide sequences in such a way that, when they are cloned in the vector virus, they present, in the 5′ region, nucleotides of the 5′ end of the vector virus NS1 gene or functionally equivalent sequences, and, in its 3′ end, the totality or part of the anchorage genome region (“stem anchor”) of E protein of the vector virus or functionally equivalent sequences, which do not compromise the structure and replication of said vector virus;

(b) insertion of the modified heterologous sequences as described in (a) into the intergenic region between the genes encoding E structural protein and NS1 nonstructural protein of the vector virus;

(c) regeneration of the nonpathogenic recombinant virus, having the heterologous sequences stably integrated into the viral genome at the insertion point described in (b), and expression of the heterologous sequence in such a way that the corresponding protein induces an appropriate immune response.

Thus, the inventors found that the strategy for E/NS1 insertion may be applied to generate recombinant vaccines against lentiviruses, particularly the HIV (Human Immunodeficiency Virus).

In one embodiment of a recombinant virus as contemplated herein, an attenuated recombinant virus is a yellow fever vaccinal virus 17D expressing a sequence of SIVmac239 Gag sequences which is used as a viral vector to originate SIV-specific CD8+ T-cell responses in rhesus monkeys. The model of Asian rhesus monkey was employed to validate the expression of HIV antigens in the yellow fever 17D virus vector because the SIV-infected rhesus monkey model reproduces, in many aspects, the immunodeficiency that occurs in HIV-infected humans. First, several SIV strains are closely related to HIV-1 strains and infect CD4+ T cells, giving rise to a decline in their counts over time. SIV infection causes a disease that is similar to AIDS in most of the monkeys infected for one year after inoculation. Moreover, many genes of the monkeys which encode key proteins in the immune system are similar to those of humans. Equivalents of the genes for HLA class, class II [sic] and T-cell receptor (TCR) are found in the monkeys. Orthologs of HLA-A, -B, -E and -F locations were isolated in rhesus monkeys. In both infections, CD8+ T cells select the major part of the sequence variation outside the envelope protein and the natural control of viral replication is associated with certain MHC-I alleles [11,12].

Consequently, as disclosed herein, the vaccination of rhesus monkeys with different formulations containing recombinant yellow fever virus was assessed so as to determine the potential use of 17D vector in the development of new vaccines against HIV. The recombinant yellow fever virus obtained expresses a SIVmac239 Gag fragment encompassing amino acids 45 to 269. Gag is a precursory protein and, in the course of viral replication, it undergoes proteolytic splitting by the viral protease, producing the internal structural proteins of the mature virion. It was determined that multiple Gag-specific responses are important in the control of SIV replication. This may be due to infected cells which present Gag-derived CD8+ T-cell epitopes right after the viral entry. Gag-specific CD8+ T cells recognize infected cells in up to 2 hours after the infection. Six hours after the infection, Gag-specific CD8+ T cells eliminate the infected cells, both before and after the integration of proviral DNA and viral protein synthesis. These findings demonstrate that Gag-specific CD8+ T cells do not require productive cell infection or even protein synthesis “again” to recognize the infected cells and eliminate them. This suggests that Gag-specific CD8+ T cells may be much more important than previously imagined.

In order to reach the goals disclosed herein, the following sequences were used:

SEQ. ID. NO.: 1

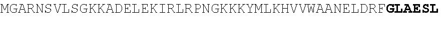

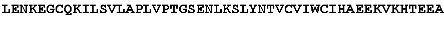

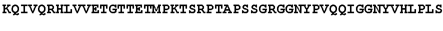

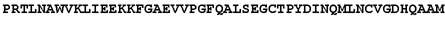

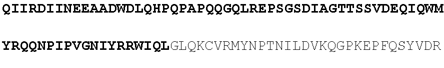

FYKSLRAEQTDAAVKNWMTQTLLIQNANPDCKLVLKGLGVNPTLEEMLTA
CQGVGGPGQKARLMAEALKEALAPVPIPFAAAQQRGPRKTIKCWNCGKEG
HSARQCRAPRRQGCWKCGKMDHVMAKCPDRQAGFLGLGPWGKKPRNFPMA
QVHQGLMPTAPPEDPAVDLLKNYMQLGKQQREKQRESRXKPYKEVTEDLL
HLNSLFGGDQ.

(TO BE CONFIRMED, but in several seqs. of the GENBANK submitted by David's group, the seqs. have 510 amino acids—the final Q residue is missing).

SEQ. ID. NO.: 2 is a sequence of amino acids of the recombinant protein Gag 45-269, cloned and expressed in the E/NS1 intergenic region by the yellow fever 17D virus. Gag45-269 fragment is shaded in gray.

SEQ. ID. NO.: 2

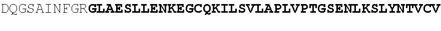

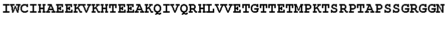

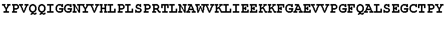

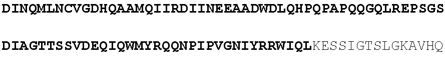

VFGSVYTTMFGGVSWMIRILIGFLVLWIGTNSRNTSMAMTCIAVGGITLF
LGFTVGA

Wherein the other sequences present in SEQ. ID. NO.: 2 are sequences of the yellow fever virus and correspond to:

SEQ. ID. NO.: 3

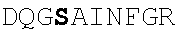

SEQ. ID. NO.: 3 is a variation of 10 amino acid residues of the N-terminal sequence of NS1 protein of the yellow fever virus (DQG [symbol] AINFGK); YF genome position from 2453 to 2482)[sic] in which an amino acid replacement was introduced (from cysteine to serine, shaded in gray).

SEQ. ID. NO.: 4
KESSIG

SEQ. ID. NO.: 4 is the sequence of amino acids corresponding both to the sequence of nucleotides of the position from 2145 to 2164 of the yellow fever virus genome and the motif comprising the amino acid residues 391 to 397 of E protein of the yellow fever virus.

SEQ. ID. NO.: .5
TSLGKAVHQVFGSVYTTMFGGVSWMIRILIGFLVLWIGTNSRNTSMAMTC
IAVGGITLFLGFTVGA

SEQ. ID. NO.: 5 is a variation of the last-terminal [sic] amino acid residues of E protein of dengue virus serotype 4 (dengue virus serotype 4 genome position from 2226 to 2423; GenBank AF326825). SEQ. ID. NO.: 5 includes a replacement relative to the original sequence where the amino acid terminal sequence VQA is changed to VGA.

SEQ. ID. NO.: 6 is a nucleotide sequence (924 nucleotides) of the cassette of recombinant Gag45-269, cloned in the E/NS1 intergenic region of the yellow fever 17D virus. Gag 45-269 fragment is shaded in gray.

SEQ. ID. NO.: 6

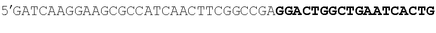

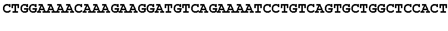

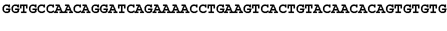

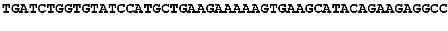

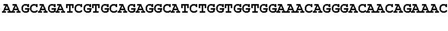

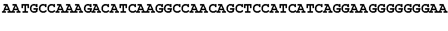

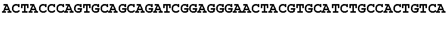

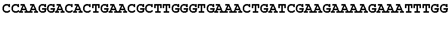

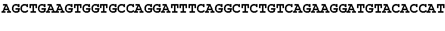

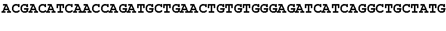

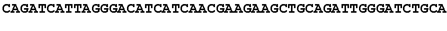

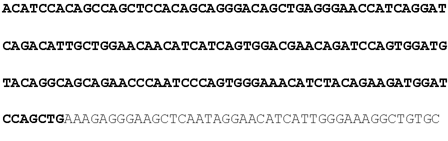

ACCAGGTTTTTGGAAGTGTGTATACAACCATGTTTGGAGGAGTCTCATGG
ATGATTAGAATCCTAATTGGGTTCTTAGTGTTGTGGATTGGCACGAACTC
CAGGAACACTTCAATGGCTATGACGTGCATAGCTGTTGGAGGAATCACTC
TGTTTCTGGGCTTCACAGTTGGCGCC 3′

SEQ. ID. NO.: 7 is a sequence of 11, 785 [sic] nucleotides of the recombinant virus genome taking cassette Gag 45-269, called rYF17D/SIVGag45-269.

SEQ. ID. NO.: 7
5′
AGTAAATCCTGTGTGCTAATTGAGGTGCATTGGTCTGCAAATCGAGTTGC
TAGGCAATAAACACATTTGGATTAATTTTAATCGTTCGTTGAGCGATTAG
CAGAGAACTGACCAGAACATGTCTGGTCGTAAAGCTCAGGGAAAAACCCT
GGGCGTCAATATGGTACGACGAGGAGTTCGCTCCTTGTCAAACAAAATAA
AACAAAAAACAAAACAAATTGGAAACAGACCTGGACCTTCAAGAGGTGTT
CAAGGATTTATCTTTTTCTTTTTGTTCAACATTTTGACTGGAAAAAAGAT
CACAGCCCACCTAAAGAGGTTGTGGAAAATGCTGGACCCAAGACAAGGCT
TGGCTGTTCTAAGGAAAGTCAAGAGAGTGGTGGCCAGTTTGATGAGAGGA
TTGTCCTCAAGGAAACGCCGTTCCCATGATGTTCTGACTGTGCAATTCCT
AATTTTGGGAATGCTGTTGATGACCGGTGGAGTGACCTTGGTGCGGAAAA
ACAGATGGTTGCTCCTAAATGTGACATCTGAGGACCTCGGGAAAACATTC
TCTGTGGGCACAGGCAACTGCACAACAAACATTTTGGAAGCCAAGTACTG
GTGCCCAGACTCAATGGAATACAACTGTCCCAATCTCAGTCCAAGAGAGG
AGCCAGATGACATTGATTGCTGGTGCTATGGGGTGGAAAACGTTAGAGTC
GCATATGGTAAGTGTGACTCAGCAGGCAGGTCTAGGAGGTCAAGAAGGGC
CATTGACTTGCCTACGCATGAAAACCATGGTTTGAAGACCCGGCAAGAAA
AATGGATGACTGGAAGAATGGGTGAAAGGCAACTCCAAAAGATTGAGAGA
TGGTTCGTGAGGAACCCCTTTTTTGCAGTGACGGCTCTGACCATTGCCTA
CCTTGTGGGAAGCAACATGACGCAACGAGTCGTGATTGCCCTACTGGTCT
TGGCTGTTGGTCCGGCCTACTCAGCTCACTGCATTGGAATTACTGACAGG
GATTTCATTGAGGGGGTGCATGGAGGAACTTGGGTTTCAGCTACCCTGGA
GCAAGACAAGTGTGTCACTGTTATGGCCCCTGACAAGCCTTCATTGGACA
TCTCACTAGAGACAGTAGCCATTGATAGACCTGCTGAGGCGAGGAAAGTG
TGTTACAATGCAGTTCTCACTCATGTGAAGATTAATGACAAGTGCCCCAG
CACTGGAGAGGCCCACCTAGCTGAAGAGAACGAAGGGGACAATGCGTGCA
AGCGCACTTATTCTGATAGAGGCTGGGGCAATGGCTGTGGCCTATTTGGG
AAAGGGAGCATTGTGGCATGCGCCAAATTCACTTGTGCCAAATCCATGAG
TTTGTTTGAGGTTGATCAGACCAAAATTCAGTATGTCATCAGAGCACAAT
TGCATGTAGGGGCCAAGCAGGAAAATTGGAATACCAGCATTAAGACTCTC
AAGTTTGATGCCCTGTCAGGCTCCCAGGAAGTCGAGTTCATTGGGTATGG
AAAAGCTACACTGGAATGCCAGGTGCAAACTGCGGTGGACTTTGGTAACA
GTTACATCGCTGAGATGGAAACAGAGAGCTGGATAGTGGACAGACAGTGG
GCCCAGGACTTGACCCTGCCATGGCAGAGTGGAAGTGGCGGGGTGTGGAG
AGAGATGCATCATCTTGTCGAATTTGAACCTCCGCATGCCGCCACTATCA
GAGTACTGGCCCTGGGAAACCAGGAAGGCTCCTTGAAAACAGCTCTTACT
GGCGCAATGAGGGTTACAAAGGACACAAATGACAACAACCTTTACAAACT
ACATGGTGGACATGTTTCTTGCAGAGTGAAATTGTCAGCTTTGACACTCA
AGGGGACATCCTACAAAATATGCACTGACAAAATGTTTTTTGTCAAGAAC
CCAACTGACACTGGCCATGGCACTGTTGTGATGCAGGTGAAAGTGTCAAA
AGGAACCCCCTGCAGGATTCCAGTGATAGTAGCTGATGATCTTACAGCGG
CAATCAATAAAGGCATTTTGGTTACAGTTAACCCCATCGCCTCAACCAAT
GATGATGAAGTGCTGATTGAGGTGAACCCACCTTTTGGAGACAGCTACAT
TATCGTTGGGAGAGGAGATTCACGTCTCACTTACCAGTGGCACAAAGAGG
GAAGCTCAATAGGAAAGTTGTTCACTCAGACCATGAAAGGCGTGGAACGC
CTGGCCGTCATGGGAGACACCGCCTGGGATTTCAGCTCCGCTGGAGGGTT
CTTCACTTCGGTTGGGAAAGGAATTCATACGGTGTTTGGCTCTGCCTTTC
AGGGGCTATTTGGCGGCTTGAACTGGATAACAAAGGTCATCATGGGGGCG
GTACTCATATGGGTTGGCATCAACACAAGAAACATGACAATGTCCATGAG
CATGATCTTGGTAGGAGTGATCATGATGTTTTTGTCTCTAGGAGTTGGGG

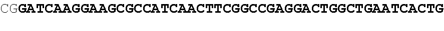

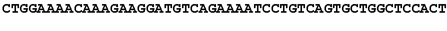

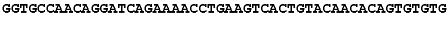

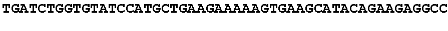

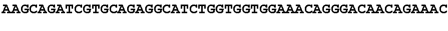

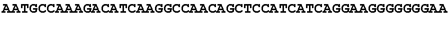

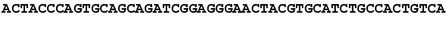

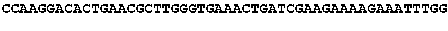

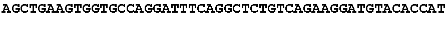

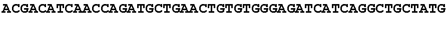

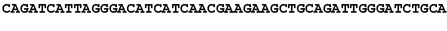

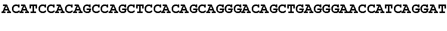

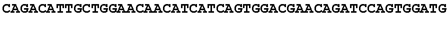

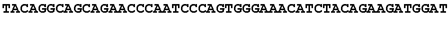

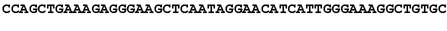

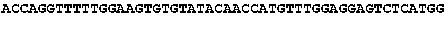

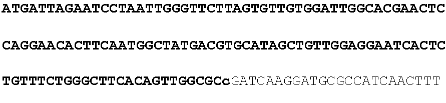

GGCAAGAGAGAGCTCAAGTGCGGAGATGGTATCTTCATATTTAGAGACTCT
GATGACTGGCTGAACAAGTACTCATACTATCCAGAAGATCCTGTGAAGCTT
GCATCAATAGTGAAAGCCTCTTTCGAAGAAGGGAAGTGTGGCCTAAATTCA
GTTGACTCCCTTGAGCATGAGATGTGGAGAAGCAGGGCAGATGAGATTAAT
ACCATTTTTGAGGAAAACGAGGTGGACATTTCTGTTGTCGTGCAGGATCCA
AAGAATGTTTACCAGAGAGGAACTCATCCATTTTCCAGAATTCGGGATGGT
CTGCAGTATGGTTGGAAGACTTGGGGTAAGAACCTTGTGTTCTCCCCAGGG
AGGAAGAATGGAAGCTTCATCATAGATGGAAAGTCCAGGAAAGAATGCCCG
TTTTCAAACCGGGTCTGGAATTCTTTCCAGATAGAGGAGTTTGGGACGGGA
GTGTTCACCACACGCGTGTACATGGACGCAGTCTTTGAATACACCATAGAC
TGCGATGGATCTATCTTGGGTGCAGCGGTGAACGGAAAAAAGAGTGCCCAT
GGCTCTCCAACATTTTGGATGGGAAGTCATGAAGTAAATGGGACATGGATG
ATCCACACCTTGGAGGCATTAGATTACAAGGAGTGTGAGTGGCCACTGACA
CATACGATTGGAACATCAGTTGAAGAGAGTGAAATGTTCATGCCGAGATCA
ATCGGAGGCCCAGTTAGCTCTCACAATCATATCCCTGGATACAAGGTTCAG
ACGAACGGACCTTGGATGCAGGTACCACTAGAAGTGAAGAGAGAAGCTTGC
CCAGGGACTAGCGTGATCATTGATGGCAACTGTGATGGACGGGGAAAATCA
ACCAGATCCACCACGGATAGCGGGAAAGTTATTCCTGAATGGTGTTGCCGC
TCCTGCACAATGCCGCCTGTGAGCTTCCATGGTAGTGATGGGTGTTGGTAT
CCCATGGAAATTAGGCCAAGGAAAACGCATGAAAGCCATCTGGTGCGCTCC
TGGGTTACAGCTGGAGAAATACATGCTGTCCCTTTTGGTTTGGTGAGCATG
ATGATAGCAATGGAAGTGGTCCTAAGGAAAAGACAGGGACCAAAGCAAATG
TTGGTTGGAGGAGTAGTGCTCTTGGGAGCAATGCTGGTCGGGCAAGTAACT
CTCCTTGATTTGCTGAAACTCACAGTGGCTGTGGGATTGCATTTCCATGAG
ATGAACAATGGAGGAGACGCCATGTATATGGCGTTGATTGCTGCCTTTTCA
ATCAGACCAGGGCTGCTCATCGGCTTTGGGCTCAGGACCCTATGGAGCCCT
CGGGAACGCCTTGTGCTGACCCTAGGAGCAGCCATGGTGGAGATTGCCTTG
GGTGGCGTGATGGGCGGCCTGTGGAAGTATCTAAATGCAGTTTCTCTCTGC
ATCCTGACAATAAATGCTGTTGCTTCTAGGAAAGCATCAAATACCATCTTG
CCCCTCATGGCTCTGTTGACACCTGTCACTATGGCTGAGGTGAGACTTGCC
GCAATGTTCTTTTGTGCCATGGTTATCATAGGGGTCCTTCACCAGAATTTC
AAGGACACCTCCATGCAGAAGACTATACCTCTGGTGGCCCTCACACTCACA
TCTTACCTGGGCTTGACACAACCTTTTTTGGGCCTGTGTGCATTTCTGGCA
ACCCGCATATTTGGGCGAAGGAGTATCCCAGTGAATGAGGCACTCGCAGCA
GCTGGTCTAGTGGGAGTGCTGGCAGGACTGGCTTTTCAGGAGATGGAGAAC
TTCCTTGGTCCGATTGCAGTTGGAGGACTCCTGATGATGCTGGTTAGCGTG
GCTGGGAGGGTGGATGGGCTAGAGCTCAAGAAGCTTGGTGAAGTTTCATGG
GAAGAGGAGGCGGAGATCAGCGGGAGTTCCGCCCGCTATGATGTGGCACTC
AGTGAACAAGGGGAGTTCAAGCTGCTTTCTGAAGAGAAAGTGCCATGGGAC
CAGGTTGTGATGACCTCGCTGGCCTTGGTTGGGGCTGCCCTCCATCCATTT
GCTCTTCTGCTGGTCCTTGCTGGGTGGCTGTTTCATGTCAGGGGAGCTAGG
AGAAGTGGGGATGTCTTGTGGGATATTCCCACTCCTAAGATCATCGAGGAA
TGTGAACATCTGGAGGATGGGATTTATGGCATATTCCAGTCAACCTTCTTG
GGGGCCTCCCAGCGAGGAGTGGGAGTGGCACAGGGAGGGGTGTTCCACACA
ATGTGGCATGTCACAAGAGGAGCTTTCCTTGTCAGGAATGGCAAGAAGTTG
ATTCCATCTTGGGCTTCAGTAAAGGAAGACCTTGTCGCCTATGGTGGCTCA
TGGAAGTTGGAAGGCAGATGGGATGGAGAGGAAGAGGTCCAGTTGATCGCG
GCTGTTCCAGGAAAGAACGTGGTCAACGTCCAGACAAAACCGAGCTTGTTC
AAAGTGAGGAATGGGGGAGAAATCGGGGCTGTCGCTCTTGACTATCCGAGT
GGCACTTCAGGATCTCCTATTGTTAACAGGAACGGAGAGGTGATTGGGCTG
TACGGCAATGGCATCCTTGTCGGTGACAACTCCTTCGTGTCCGCCATATCC
CAGACTGAGGTGAAGGAAGAAGGAAAGGAGGAGCTCCAAGAGATCCCGACA
ATGCTAAAGAAAGGAATGACAACTGTCCTTGATTTTCATCCTGGAGCTGGG
AAGACAAGACGTTTCCTCCCACAGATCTTGGCCGAGTGCGCACGGAGACGC
TTGCGCACTCTTGTGTTGGCCCCCACCAGGGTTGTTCTTTCTGAAATGAAG
GAGGCTTTTCACGGCCTGGACGTGAAATTCCACACACAGGCTTTTTCCGCT
CACGGCAGCGGGAGAGAAGTCATTGATGCCATGTGCCATGCCACCCTAACT
TACAGGATGTTGGAACCAACTAGGGTTGTTAACTGGGAAGTGATCATTATG
GATGAAGCCCATTTTTTGGATCCAGCTAGCATAGCCGCTAGAGGTTGGGCA
GCGCACAGAGCTAGGGCAAATGAAAGTGCAACAATCTTGATGACAGCCACA
CCGCCTGGGACTAGTGATGAATTTCCACATTCAAATGGTGAAATAGAAGAT
GTTCAAACGGACATACCCAGTGAGCCCTGGAACACAGGGCATGACTGGATC
CTGGCTGACAAAAGGCCCACGGCATGGTTCCTTCCATCCATCAGAGCTGCA
AATGTCATGGCTGCCTCTTTGCGTAAGGCTGGAAAGAGTGTGGTGGTCCTG
AACAGGAAAACCTTTGAGAGAGAATACCCCACGATAAAGCAGAAGAAACCT
GACTTTATATTGGCCACTGACATAGCTGAAATGGGAGCCAACCTTTGCGTG
GAGCGAGTGCTGGATTGCAGGACGGCTTTTAAGCCTGTGCTTGTGGATGAA
GGGAGGAAGGTGGCAATAAAAGGGCCACTTCGTATCTCCGCATCCTCTGCT
GCTCAAAGGAGGGGGCGCATTGGGAGAAATCCCAACAGAGATGGAGACTCA
TACTACTATTCTGAGCCTACAAGTGAAAATAATGCCCACCACGTCTGCTGG
TTGGAGGCCTCAATGCTCTTGGACAACATGGAGGTGAGGGGTGGAATGGTC
GCCCCACTCTATGGCGTTGAAGGAACTAAAACACCAGTTTCCCCTGGTGAA
ATGAGACTGAGGGATGACCAGAGGAAAGTCTTCAGAGAACTAGTGAGGAAT
TGTGACCTGCCCGTTTGGCTTTCGTGGCAAGTGGCCAAGGCTGGTTTGAAG
ACGAATGATCGTAAGTGGTGTTTTGAAGGCCCTGAGGAACATGAGATCTTG
AATGACAGCGGTGAAACAGTGAAGTGCAGGGCTCCTGGAGGAGCAAAGAAG
CCTCTGCGCCCAAGGTGGTGTGATGAAAGGGTGTCATCTGACCAGAGTGCG
CTGTCTGAATTTATTAAGTTTGCTGAAGGTAGGAGGGGAGCTGCTGAAGTG
CTAGTTGTGCTGAGTGAACTCCCTGATTTCCTGGCTAAAAAAGGTGGAGAG
GCAATGGATACCATCAGTGTGTTCCTCCACTCTGAGGAAGGCTCTAGGGCT
TACCGCAATGCACTATCAATGATGCCTGAGGCAATGACAATAGTCATGCTG
TTTATACTGGCTGGACTACTGACATCGGGAATGGTCATCTTTTTCATGTCT
CCCAAAGGCATCAGTAGAATGTCTATGGCGATGGGCACAATGGCCGGCTGT
GGATATCTCATGTTCCTTGGAGGCGTCAAACCCACTCACATCTCCTATGTC
ATGCTCATATTCTTTGTCCTGATGGTGGTTGTGATCCCCGAGCCAGGGCAA
CAAAGGTCCATCCAAGACAACCAAGTGGCATACCTCATTATTGGCATCCTG
ACGCTGGTTTCAGCGGTGGCAGCCAACGAGCTAGGCATGCTGGAGAAAACC
AAAGAGGACCTCTTTGGGAAGAAGAACTTAATTCCATCTAGTGCTTCACCC
TGGAGTTGGCCGGATCTTGACCTGAAGCCAGGAGCTGCCTGGACAGTGTAC
GTTGGCATTGTTACAATGCTCTCTCCAATGTTGCACCACTGGATCAAAGTC
GAATATGGCAACCTGTCTCTGTCTGGAATAGCCCAGTCAGCCTCAGTCCTT
TCTTTCATGGACAAGGGGATACCATTCATGAAGATGAATATCTCGGTCATA
ATGCTGCTGGTCAGTGGCTGGAATTCAATAACAGTGATGCCTCTGCTCTGT
GGCATAGGGTGCGCCATGCTCCACTGGTCTCTCATTTTACCTGGAATCAAA
GCGCAGCAGTCAAAGCTTGCACAGAGAAGGGTGTTCCATGGCGTTGCCAAG
AACCCTGTGGTTGATGGGAATCCAACAGTTGACATTGAGGAAGCTCCTGAA
ATGCCTGCCCTTTATGAGAAGAAACTGGCTCTATATCTCCTTCTTGCTCTC
AGCCTAGCTTCTGTTGCCATGTGCAGAACGCCCTTTTCATTGGCTGAAGGC
ATTGTCCTAGCATCAGCTGCCTTAGGGCCGCTCATAGAGGGAAACACCAGC
CTTCTTTGGAATGGACCCATGGCTGTCTCCATGACAGGAGTCATGAGGGGG
AATCACTATGCTTTTGTGGGAGTCATGTACAATCTATGGAAGATGAAAACT
GGACGCCGGGGGAGCGCGAATGGAAAAACTTTGGGTGAAGTCTGGAAGAGG
GAACTGAATCTGTTGGACAAGCGACAGTTTGAGTTGTATAAAAGGACCGAC
ATTGTGGAGGTGGATCGTGATACGGCACGCAGGCATTTGGCCGAAGGGAAG
GTGGACACCGGGGTGGCGGTCTCCAGGGGGACCGCAAAGTTAAGGTGGTTC
CATGAGCGTGGCTATGTCAAGCTGGAAGGTAGGGTGATTGACCTGGGGTGT
GGCCGCGGAGGCTGGTGTTACTACGCTGCTGCGCAAAAGGAAGTGAGTGGG
GTCAAAGGATTTACTCTTGGAAGAGACGGCCATGAGAAACCCATGAATGTG
CAAAGTCTGGGATGGAACATCATCACCTTCAAGGACAAAACTGATATCCAC
CGCCTAGAACCAGTGAAATGTGACACCCTTTTGTGTGACATTGGAGAGTCA
TCATCGTCATCGGTCACAGAGGGGGAAAGGACCGTGAGAGTTCTTGATACT
GTAGAAAAATGGCTGGCTTGTGGGGTTGACAACTTCTGTGTGAAGGTGTTA
GCTCCATACATGCCAGATGTTCTTGAGAAACTGGAATTGCTCCAAAGGAGG
TTTGGCGGAACAGTGATCAGGAACCCTCTCTCCAGGAATTCCACTCATGAA
ATGTACTACGTGTCTGGAGCCCGCAGCAATGTCACATTTACTGTGAACCAA
ACATCCCGCCTCCTGATGAGGAGAATGAGGCGTCCAACTGGAAAAGTGACC
CTGGAGGCTGACGTCATCCTCCCAATTGGGACACGCAGTGTTGAGACAGAC
AAGGGACCCCTGGACAAAGAGGCCATAGAAGAAAGGGTTGAGAGGATAAAA
TCTGAGTACATGACCTCTTGGTTTTATGACAATGACAACCCCTACAGGACC
TGGCACTACTGTGGCTCCTATGTCACAAAAACCTCAGGAAGTGCGGCGAGC
ATGGTAAATGGTGTTATTAAAATTCTGACATATCCATGGGACAGGATAGAG
GAGGTCACCAGAATGGCAATGACTGACACAACCCCTTTTGGACAGCAAAGA
GTGTTTAAAGAAAAAGTTGACACCAGAGCAAAGGATCCACCAGCGGGAACT
AGGAAGATCATGAAAGTTGTCAACAGGTGGCTGTTCCGCCACCTGGCCAGA
GAAAAGAGCCCCAGACTGTGCACAAAGGAAGAATTTATTGCAAAAGTCCGA
AGTCATGCAGCCATTGGAGCTTACCTGGAAGAACAAGAACAGTGGAAGACT
GCCAATGAGGCTGTCCAAGACCCAAAGTTCTGGGAACTGGTGGATGAAGAA
AGGAAGCTGCACCAACAAGGCAGGTGTCGGACTTGTGTGTACAACATGATG
GGGAAAAGAGAGAAGAAGCTGTCAGAGTTTGGGAAAGCAAAGGGAAGCCGT
GCCATATGGTATATGTGGCTGGGAGCGCGGTATCTTGAGTTTGAGGCCCTG
GGATTCCTGAATGAGGACCATTGGGCTTCCAGGGAAAACTCAGGAGGAGGA
GTGGAAGGCATTGGCTTACAATACCTAGGATATGTGATCAGAGACCTGGCT
GCAATGGATGGTGGTGGATTCTACGCGGATGACACCGCTGGATGGGACACG
CGCATCACAGAGGCAGACCTTGATGATGAACAGGAGATCTTGAACTACATG
AGCCCACATCACAAAAAACTGGCACAAGCAGTGATGGAAATGACATACAAG
AACAAAGTGGTGAAAGTGTTGAGACCAGCCCCAGGAGGGAAAGCCTACATG
GATGTCATAAGTCGACGAGACCAGAGAGGATCCGGGCAGGTAGTGACTTAT
GCTCTGAACACCATCACCAACTTGAAAGTCCAATTGATCAGAATGGCAGAA
GCAGAGATGGTGATACATCACCAACATGTTCAAGATTGTGATGAATCAGTT
CTGACCAGGCTGGAGGCATGGCTCACTGAGCACGGATGTAACAGACTGAAG
AGGATGGCGGTGAGTGGAGACGACTGTGTGGTCCGGCCCATCGATGACAGG
TTCGGCCTGGCCCTGTCCCATCTCAACGCCATGTCCAAGGTTAGAAAGGAC
ATATCTGAATGGCAGCCATCAAAAGGGTGGAATGATTGGGAGAATGTGCCC
TTCTGTTCCCACCACTTCCATGAACTACAGCTGAAGGATGGCAGGAGGATT
GTGGTGCCTTGCCGAGAACAGGACGAGCTCATTGGGAGAGGAAGGGTGTCT
CCAGGAAACGGCTGGATGATCAAGGAAACAGCTTGCCTCAGCAAAGCCTAT
GCCAACATGTGGTCACTGATGTATTTTCACAAAAGGGACATGAGGCTACTG
TCATTGGCTGTTTCCTCAGCTGTTCCCACCTCATGGGTTCCACAAGGACGC
ACAACATGGTCGATTCATGGGAAAGGGGAGTGGATGACCACGGAAGACATG
CTTGAGGTGTGGAACAGAGTATGGATAACCAACAACCCACACATGCAGGAC
AAGACAATGGTGAAAAAATGGAGAGATGTCCCTTATCTAACCAAGAGACAA
GACAAGCTGTGCGGATCACTGATTGGAATGACCAATAGGGCCACCTGGGCC
TCCCACATCCATTTAGTCATCCATCGTATCCGAACGCTGATTGGACAGGAG
AAATACACTGACTACCTAACAGTCATGGACAGGTATTCTGTGGATGCTGAC
CTGCAACTGGGTGAGCTTATCTGAAACACCATCTAACAGGAATAACCGGGA
TACAAACCACGGGTGGAGAACCGGACTCCCCACAACCTGAAACCGGGATAT
AAACCACGGCTGGAGAACCGGGCTCCGCACTTAAAATGAAACAGAAACCGG
GATAAAAACTACGGATGGAGAACCGGACTCCACACATTGAGACAGAAGAAG
TTGTCAGCCCAGAACCCCACACGAGTTTTGCCACTGCTAAGCTGTGAGGCA
GTGCAGGCTGGGACAGCCGACCTCCAGGTTGCGAAAAACCTGGTTTCTGGG
ACCTCCCACCCCAGAGTAAAAAGAACGGAGCCTCCGCTACCACCCTCCCAC
GTGGTGGTAGAAAGACGGGGTCTAGAGGTTAGAGGAGACCCTCCAGGGAAC
AAATAGTGGGACCATATTGACGCCAGGGAAAGACCGGAGTGGTTCTCTGCT
TTTCCTCCAGAGGTCTGTGAGCACAGTTTGCTCAAGAATAAGCAGACCTTT
GGATGACAAACACAAAACCAC 3′

SEQ. ID. NO.: 8 is a precursory sequence of the polyprotein of virus rYF17D/SIVGag45-269. The recombinant protein is shaded in gray.

SEQ. ID. NO.: 8
SGRKAQGKTLGVNMVRRGVRSLSNKIKQKTKQIGNRPGPSRGVQGFIFFF
LFNILTGKKITAHLKRLWKMLDPRQGLAVLRKVKRVVASLMRGLSSRKRR
SHDVLTVQFLILGMLLMTGGVTLVRKNRWLLLNVTSEDLGKTFSVGTGNC
TTNILEAKYWCPDSMEYNCPNLSPREEPDDIDCWCYGVENVRVAYGKCDS
AGRSRRSRRAIDLPTHENHGLKTRQEKWMTGRMGERQLQKIERWFVRNPF
FAVTALTIAYLVGSNMTQRVVIALLVLAVGPAYSAHCIGITDRDFIEGVH
GGTWVSATLEQDKCVTVMAPDKPSLDISLETVAIDRPAEARKVCYNAVLT
HVKINDKCPSTGEAHLAEENEGDNACKRTYSDRGWGNGCGLFGKGSIVAC
AKFTCAKSMSLFEVDQTKIQYVIRAQLHVGAKQENWNTSIKTLKFDALSG
SQEVEFIGYGKATLECQVQTAVDFGNSYIAEMETESWIVDRQWAQDLTLP
WQSGSGGVWREMHHLVEFEPPHAATIRVLALGNQEGSLKTALTGAMRVTK
DTNDNNLYKLHGGHVSCRVKLSALTLKGTSYKICTDKMFFVKNPTDTGHG
TVVMQVKVSKGTPCRIPVIVADDLTAAINKGILVTVNPIASTNDDEVLIE
VNPPFGDSYTIVGRGDSRLTYQWHKEGSSIGKLFTQTMKGVERLAVMGDT
AWDFSSAGGFFTSVGKGIHTVFGSAFQGLFGGLNWITKVIMGAVLIWVGI

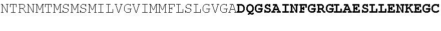

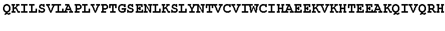

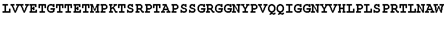

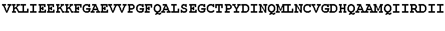

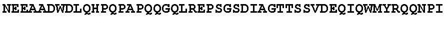

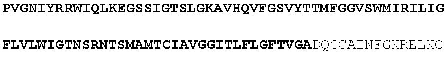

GDGIFIFRDSDDWLNKYSYYPEDPVKLASIVKASFEEGKCGLNSVDSLEH
EMWRSRADEINTIFEENEVDISVVVQDPKNVYQRGTHPFSRIRDGLQYGW
KTWGKNLVFSPGRKNGSFIIDGKSRKECPFSNRVWNSFQIEEFGTGVETT
RVYMDAVFEYTIDCDGSILGAAVNGKKSAHGSPTFWMGSHEVNGTWMIHT
LEALDYKECEWPLTHTIGTSVEESEMFMPRSIGGPVSSHNHIPGYKVQTN
GPWMQVPLEVKREACPGTSVIIDGNCDGRGKSTRSTTDSGKVIPEWCCRS
CTMPPVSFHGSDGCWYPMEIRPRKTHESHLVRSWVTAGEIHAVPFGLVSM
MIAMEVVLRKRQGPKQMLVGGVVLLGAMLVGQVTLLDLLKLTVAVGLHFH
EMNNGGDAMYMALIAAFSIRPGLLIGFGLRTLWSPRERLVLTLGAAMVEI
ALGGVMGGLWKYLNAVSLCILTINAVASRKASNTILPLMALLTPVTMAEV
RLAAMFFCAMVIIGVLHQNFKDTSMQKTIPLVALTLTSYLGLTQPFLGLC
AFLATRIFGRRSIPVNEALAAAGLVGVLAGLAFQEMENFLGPIAVGGLLM
MLVSVAGRVDGLELKKLGEVSWEEEAEISGSSARYDVALSEQGEFKLLSE
EKVPWDQVVMTSLALVGAALHPFALLLVLAGWLFHVRGARRSGDVLWDIP
TPKIIEECEHLEDGIYGIFQSTFLGASQRGVGVAQGGVFHTMWHVTRGAF
LVRNGKKLIPSWASVKEDLVAYGGSWKLEGRWDGEEEVQLIAAVPGKNVV
NVQTKPSLFKVRNGGEIGAVALDYPSGTSGSPIVNRNGEVIGLYGNGILV
GDNSFVSAISQTEVKEEGKEELQEIPTMLKKGMTTVLDFHPGAGKTRRFL
PQILAECARRRLRTLVLAPTRVVLSEMKEAFHGLDVKFHTQAFSAHGSGR
EVIDAMCHATLTYRMLEPTRVVNWEVIIMDEAHFLDPASIAARGWAAHRA
RANESATILMTATPPGTSDEFPHSNGEIEDVQTDIPSEPWNTGHDWILAD
KRPTAWFLPSIRAANVMAASLRKAGKSVVVLNRKTFEREYPTIKQKKPDF
ILATDIAEMGANLCVERVLDCRTAFKPVLVDEGRKVAIKGPLRISASSAA
QRRGRIGRNPNRDGDSYYYSEPTSENNAHHVCWLEASMLLDNMEVRGGMV
APLYGVEGTKTPVSPGEMRLRDDQRKVFRELVRNCDLPVWLSWQVAKAGL
KTNDRKWCFEGPEEHEILNDSGETVKCRAPGGAKKPLRPRWCDERVSSDQ
SALSEFIKFAEGRRGAAEVLVVLSELPDFLAKKGGEAMDTISVFLHSEEG
SRAYRNALSMMPEAMTIVMLFILAGLLTSGMVIFFMSPKGISRMSMAMGT
MAGCGYLMFLGGVKPTHISYVMLIFFVLMVVVIPEPGQQRSIQDNQVAYL
IIGILTLVSAVAANELGMLEKTKEDLFGKKNLIPSSASPWSWPDLDLKPG
AAWTVYVGIVTMLSPMLHHWIKVEYGNLSLSGIAQSASVLSFMDKGIPFM
KMNISVIMLLVSGWNSITVMPLLCGIGCAMLHWSLILPGIKAQQSKLAQR
RVFHGVAKNPVVDGNPTVDIEEAPEMPALYEKKLALYLLLALSLASVAMC
RTPFSLAEGIVLASAALGPLIEGNTSLLWNGPMAVSMTGVMRGNHYAFVG
VMYNLWKMKTGRRGSANGKTLGEVWKRELNLLDKRQFELYKRTDIVEVDR
DTARRHLAEGKVDTGVAVSRGTAKLRWFHERGYVKLEGRVIDLGCGRGGW
CYYAAAQKEVSGVKGFTLGRDGHEKPMNVQSLGWNIITFKDKTDIHRLEP
VKCDTLLCDIGESSSSSVTEGERTVRVLDTVEKWLACGVDNFCVKVLAPY
MPDVLEKLELLQRRFGGTVIRNPLSRNSTHEMYYVSGARSNVTFTVNQTS
RLLMRRMRRPTGKVTLEADVILPIGTRSVETDKGPLDKEAIEERVERIKS
EYMTSWFYDNDNPYRTWHYCGSYVTKTSGSAASMVNGVIKILTYPWDRIE
EVTRMAMTDTTPFGQQRVFKEKVDTRAKDPPAGTRKIMKVVNRWLFRHLA
REKSPRLCTKEEFIAKVRSHAATGAYLEEQEQWKTANEAVQDPKFWELVD
EERKLHQQGRCRTCVYNMMGKREKKLSEFGKAKGSRAIWYMWLGARYLEF
EALGFLNEDHWASRENSGGGVEGIGLQYLGYVIRDLAAMDGGGFYADDTA
GWDTRITEADLDDEQEILNYMSPHHKKLAQAVMEMTYKNKVVKVLRPAPG
GKAYMDVISRRDQRGSGQVVTYALNTITNLKVQLIRMAEAEMVIHHQHVQ
DCDESVLTRLEAWLTEHGCNRLKRMAVSGDDCVVRPIDDRFGLALSHLNA
MSKVRKDISEWQPSKGWNDWENVPFCSHHFHELQLKDGRRIVVPCREQDE
LIGRGRVSPGNGWMIKETACLSKAYANMWSLMYFHKRDMRLLSLAVSSAV
PTSWVPQGRTTWSIHGKGEWMTTEDMLEVWNRVWITNNPHMQDKTMVKKW
RDVPYLTKRQDKLCGSLIGMTNRATWASHIHLVIHRIRTLIGQEKYTDYL
TVMDRYSVDADLQLGEL

Other appropriate sequences to prepare the recombinant yellow fever viruses contemplated herein include corresponding sequences in HIV (for example, complete Gag polypeptide or a fragment thereof). For example, the heterologous polypeptide expressed by the recombinant yellow fever viruses considered here may comprise a fragment of SEQ. ID. NO.: 9, or a related protein (for example, a protein having at least about 50%, 60%, 70%, 80%, 90%, 95%, 96%, 97%, 98%, or 99% of amino acid sequence identity with SEQ. ID. NO.: 9).

SED. [sic] ID. NO.: 9
MGARASVLSG GKLDKWEKIR LRPGGKKTYQ LKHIVWASRE
LERFAVNPGL LETGGGCKQI LVQLQPSLQT GSEELKSLYN
AVATLYCVHQ GIEVRDTKEA LDKIEEEQNK SKKKAQQAAA
DTGNSSQVSQ NYPIVQNLQG QMVHQAISPR TLNAWVKVIE
EKAFSPEVIP MFSALSEGAT PQDLNTMLNT VGGHQAAMQM
LKETINEEAA EWDRLHPAHA GPNAPGQMRE PRGSDIAGTT
STLQEQIGWM TSNPPVPVGE IYKRWIILGL NKIVRMYSPV
SILDIRQGPK EPFRDYVDRF YKTLRAEQAS QDVKNWMTET
LLVQNANPDC KTILKALGPA ATLEEMMTAC QGVGGPSHKA
RILAEAMSQV TSPANIMMQR GNFRNQRKTI KCFNCGKEGH
LARHCRAPRK KGCWKCGREG HQMKDCTERQ ANFLGKIWPS
HKGRPGNFLQ SRPEPTAPPE ESFRFGEETT TPPQKQEPLP
SQKQETIDKD LYPLASLKSL FGNDPSLQ formatting

A fragment, as considered here, may include at least 8, 9, 10, 20, 30, 40, 50 [sic] 100, or 200 contiguous amino acids of SEQ. ID. NO.: 9. In one embodiment, the fragment comprises amino acids 45-269 of SEQ. ID. NO.: 9. The heterologous polypeptide may still comprise a terminal amino acid sequence comprising KESSIG.

### EXAMPLES

The present invention will now be described in detail by the examples presented below. It is worth emphasizing that the invention is not limited to these examples, and also includes variations and modifications within the limits allowing its action.

### Example 1

### Proliferation Properties of Recombinant rYF17D/SIVGag45-269 In Vero Cells

The growth capacity of recombinant virus rYF17D/SIVGag45-269 was assessed and compared with other two viruses, YF 17DD and YF17D/G1/2 T3 vaccine (the latter corresponding to the parental vector virus, that is, heterologous insertion). Three independent experiments of virus growth in monolayers of Vero cells were performed. All experiments were performed in low MOI of 0.02 with densities of Vero cells of 62,500 cells per cm².

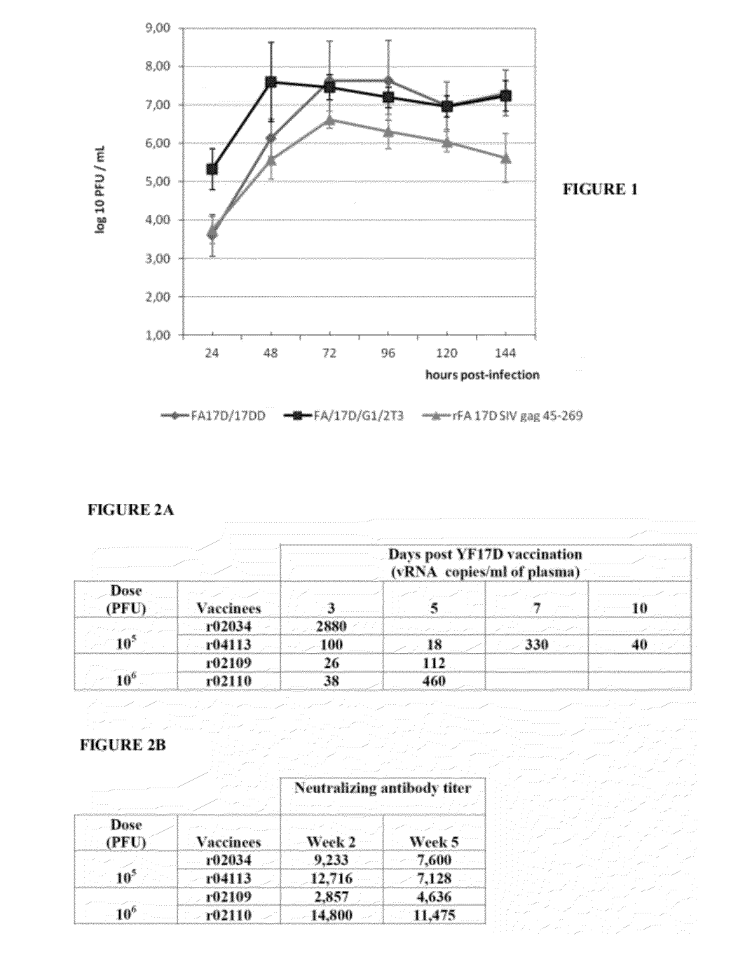

The recombinant virus rYF17D/SIVGag45-269 presented a lower growth rate compared with the viruses of 17D vaccine, and had its peak at 72 hours after the infection, exhibiting a titer of 6.61±0.23 log 10 PFU/mL. FIG. 1 shows the viral growth curves in Vero cells. Cells were infected with YF 17D control (gray lozenges), YF17D/G1/2T3 virus (black squares), or with the recombinant virus rYF17D/SIVGag45-269 (gray triangles in MOI of 0.02). Each time point represents the mean titer obtained for these three experiments separately, with the respective standard deviations.

Table 8 below shows the viral growth curves in Vero cells. Viral titers (log 10 PFU/mL) are shown in each time point. Viral growth peaks are shown in bold.

TABLE 8

| Virus | 24 h | 48 h | 72 h | 96 h | 120 h | 144 h |
|---|---|---|---|---|---|---|
| YF17D/17DD | 3.60 ± 0.54 | 6.14 ± 0.49 | 7.62 ± 1.02 | 7.63 ± 1.03 | 6.97 ± 0.62 | 7.30 ± 0.59 |
| YF/7D/G1/2T3 | 5.32 ± 0.53 | 7.60 ± 1.03 | 7.45 ± 0.33 | 7.19 ± 0.27 | 6.95 ± 0.27 | 7.23 ± 0.40 |
| rYF 17D SIV Gag | 3.74 ± 0.35 | 5.57 ± 0.51 | 6.61 ± 0.23 | 6.30 ± 0.45 | 6.04 ± 0.27 | 5.61 ± 0.64 |
| 45-269 | | | | | | |

### Example 2

### Preliminary Immunological Studies of the YF17D Vaccine Virus Infection in Mamu-A*01-Positive Rhesus Monkeys

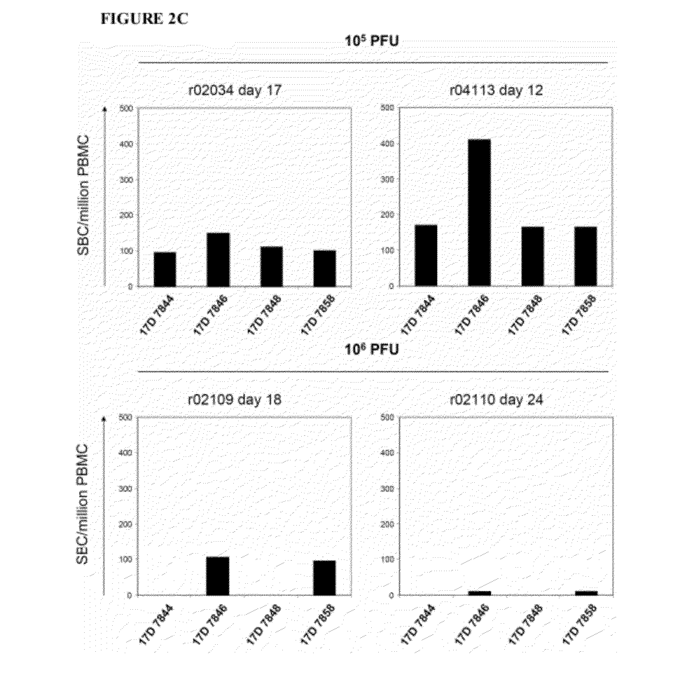

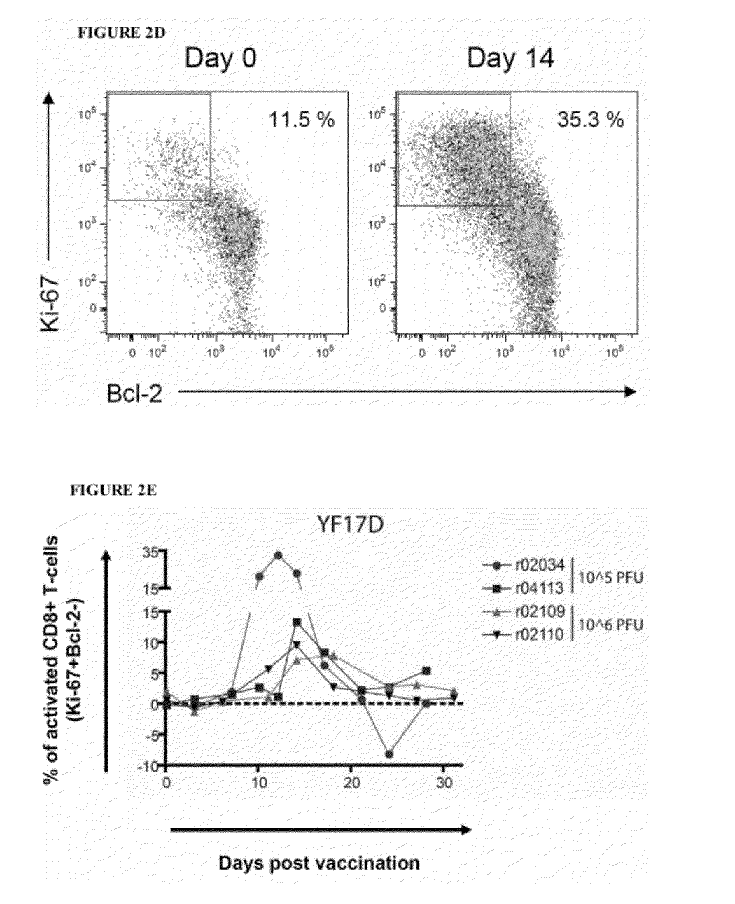

First, the YF17D vaccine virus was used to infect four Mamu-A*01-positive monkeys. The vaccine virus replicated in these four animals and induced neutralizing antibodies in all four monkeys by two weeks after vaccination (FIGS. 2A and 2B). To monitor the CD8+ T-cell immune response against YF17D, its proteome was scanned for peptides that might bind to Mamu-A*01 using algorithms (MHC Pathway) [14]. The 52 YF17D-derived peptides which were most likely to bind to Mamu-A*01, based on their predicted affinity for this MHC class I molecule, were synthesized. IFNy ELISPOT assay was used to screen these peptides in YF17D-infected animals, and four Mamu-A*01-binding peptides: 17D 7844 (LTPVTMAEV; LV91285-1293), 17D 7846 (VSPGNGWMI; V193250-3258), 17D 7850 (MSPKGISRM; MM92179-2187), and 17D 7858 (TTPFGQQRVF; TF102853-2863) were recognized in vivo (FIG. 2C). Using a previously reported protocol [15], the activation of CD8+ T cells was also observed in all four animals (FIGS. 2D and 2E). Thus, as previously observed, the YF17D vaccine virus replicates in Indian rhesus monkeys [16] and induces neutralizing antibodies, yellow fever-specific Mamu-A*01-restricted CD8+ T-cell responses, and CD8+ T-cell activation. FIG. 2 shows that YF17D replicates and induces neutralizing antibodies, virus-specific CD8+ T cells, and CD8+ T-cell activation in rhesus monkeys. FIG. 2A shows the replication of YF17D during the first 10 days after vaccination with two different doses, as measured by Q-PCR using YF17D-specific primers. FIG. 2B shows the titer of neutralizing antibodies determined at 2 and 5 weeks after YF17D vaccination. In FIG. 2C, the fresh PBMC from vaccinees (100,000 cells/well) were used in IFN-y ELISPOT assays [17] to assess T-cell responses against YF17D. Four epitopes (17D 7844, 17D 7846, 17D 7850, and 17D 7858)—predicted to bind to Mamu-A*01 as defined by the algorithm (MHC Pathway)—were selected for further studies. FIG. 2D presents the identification of activated CD8+ T cells after vaccination with YF17D based on the expression of proliferation and proapoptotic markers Ki-67 and Bcl-2 [15]. Whole blood cells were stained with antibodies against CD3 and CD8, and then permeabilized, and subsequently labeled with Bcl-2- and Ki-67-specific antibodies. The flow cytometry graphs were gated on CD3+ and CD8+ lymphocytes. FIG. E reveals the expression kinetics of Ki-67 and Bcl-2 in T cells after vaccination with YF17D.

### Example 3

### Immunogenicity of rYF17D/SIVGag45-269 in Rhesus Monkeys

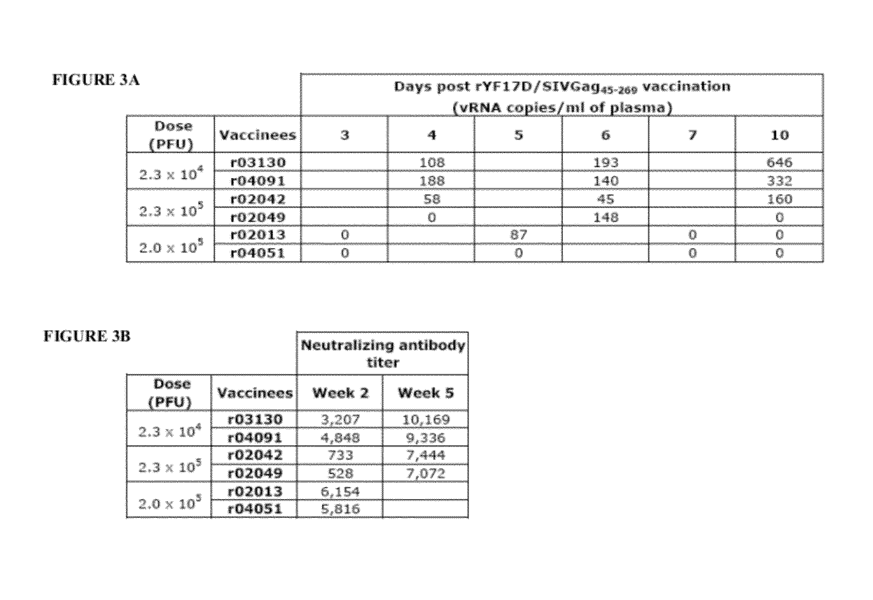

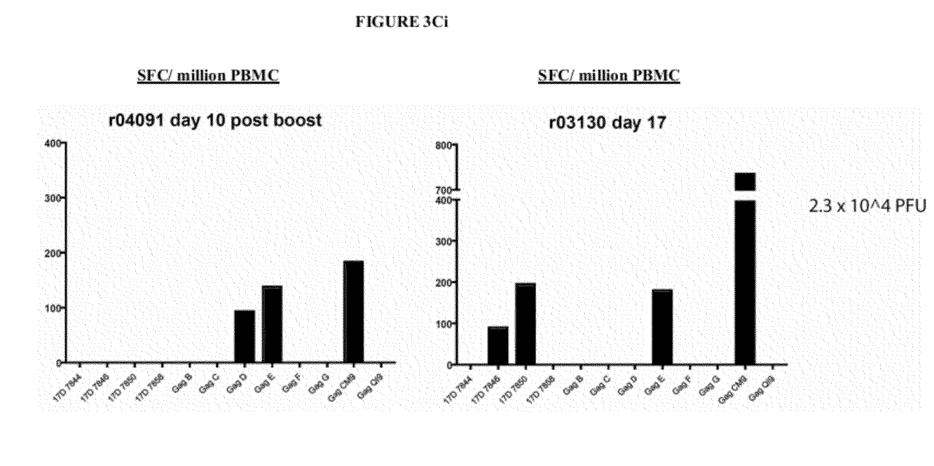

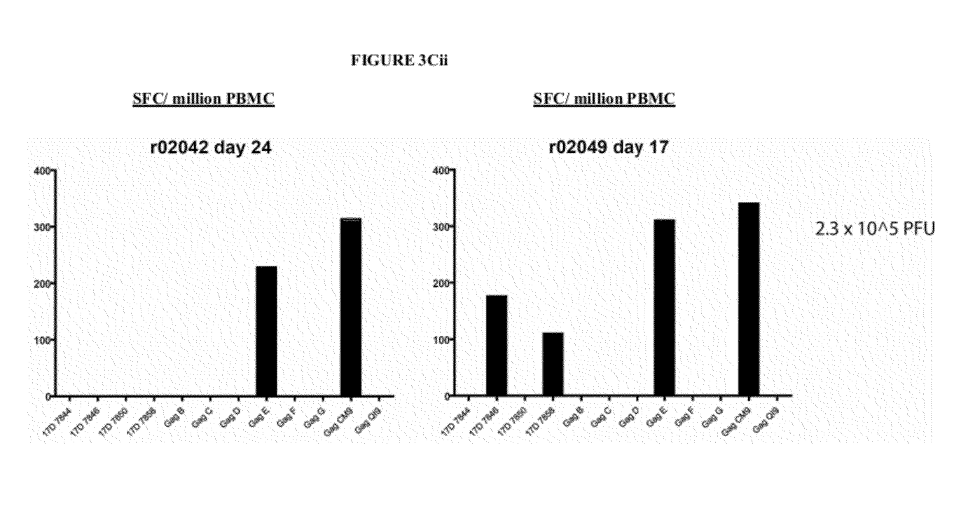

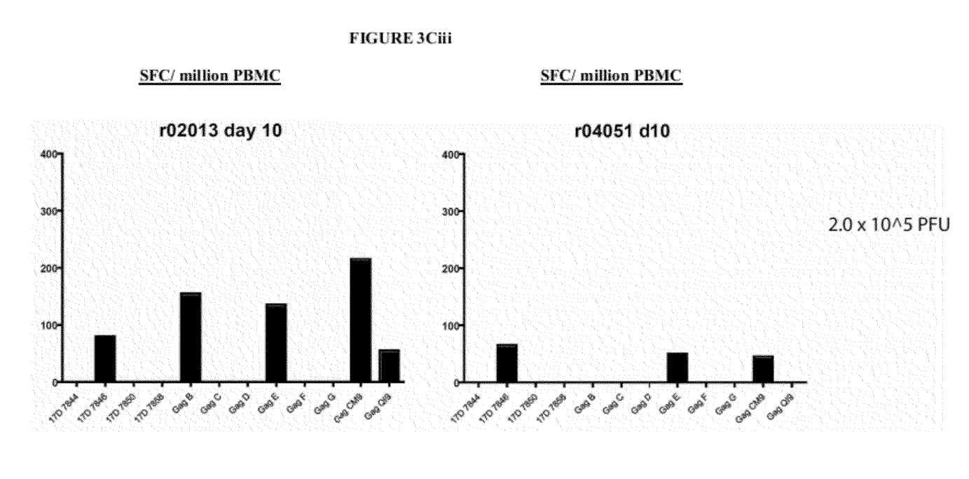

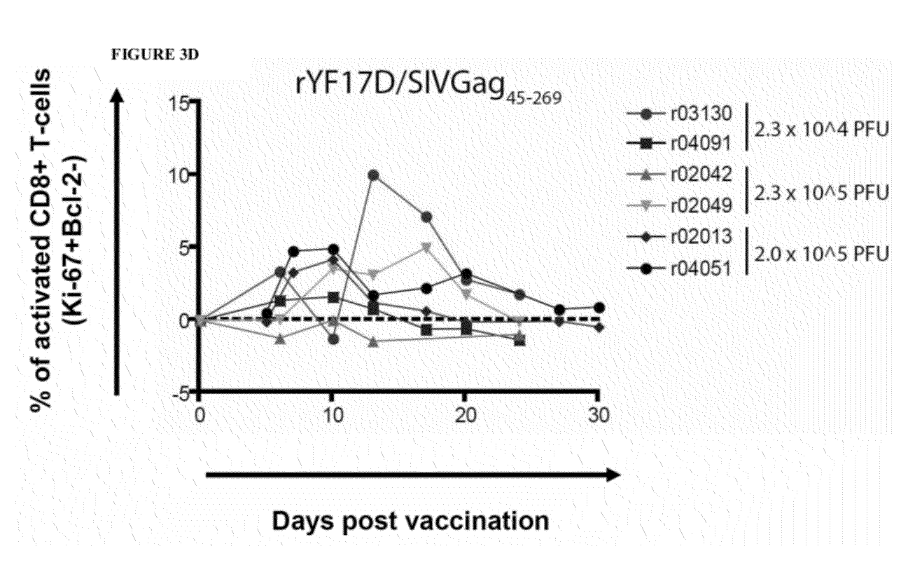

In the following step, the YF17D vaccine virus was engineered to express amino acids 45-269 of SIVmac239Gag (rYF17D/SIVGag45-269) by inserting a yellow fever codon-optimized sequence between the genes encoding the viral proteins E and NS1. This recombinant virus replicated and induced neutralizing antibodies in mice (data not shown). The rYF17D/SIVGag45-269 construct was then tested in six Mamu-A*01-positive Indian rhesus monkeys. Evidence for the viral replication of rYF17D/SIVGag45-269 was found for five of these six monkeys (FIG. 3A). Neutralizing antibodies were evident at two weeks after vaccination (FIG. 3B. [sic] A single immunization with rYF17D/SIVGag45-269 induced antigen-specific CD8+ T cells in five out of the six Mamu-A*01-positive monkeys (FIG. 3C). This recombinant virus also induced CD8+ T-cell activation in the majority of the vaccinated animals (FIG. 3D). The sixth monkey (r04091) required a booster dose 28 days after the first immunization to induce specific CD8+ T cells (data not shown and FIG. 3C). No differences were found in vaccine-induced immune responses between the animals vaccinated with YF17D and those vaccinated with rYF17D/SIVGag45-269. There was, however, considerable animal-to-animal variability. Animal r02034, vaccinated with YF17D, exhibited massive CD8+ T-cell activation (a peak of 35% at day 12; FIG. 2E), which was probably induced by the high levels of viral replication (16,800 copies/mL at day 5; FIG. 2A). The comparison of neutralizing antibody responses at 2 and 5 weeks after vaccination in animals vaccinated with YF17D or rYF17D/SIVGag45-269 did not reveal detectable differences regarding the capacity of these two vaccines to induce neutralizing antibodies (FIGS. 2B and 3B). It was also difficult to detect differences in YF17D-specific CD8+ T-cell responses induced by these two vaccines. Peak Mamu-A*01-restricted CD8+ T-cell responses against YF17D ranged from barely detectable (r02110 at day 24; FIG. 2C) to 400 SFCs/10⁶ PBMC (r04113 at day 12; FIG. 2C). Two of the animals that were vaccinated with rYF17D/SIVGag45-269 did not originate CD8+ T-cell responses against the Mamu-A*01-bound YF17D peptides, whereas this kind of response was observed in four animals (FIG. 3C), with SFCs/10⁶ PBMC ranging from 50 to 200. For almost every animal vaccinated with rYF17D/SIVGag45-269, the Gag CM9-specific responses (peak in 50-750) were higher than those generated against the Mamu-A*01-restricted YF17D epitopes (peak in 0-200), suggesting that the recombinant virus replicated in a stable manner in vivo. Thus, the recombinant YF17D virus replicated and induced both virus-specific neutralizing antibodies and CD8+ T cells that were not demonstrably different from those induced by YF17D alone.

FIG. 3 shows that rYF17D/SIVGag45-269 replicates and induces neutralizing antibodies, virus-specific CD8+ T cells, and CD8+ T-cell activation in rhesus monkeys. FIG. 3A shows the replication of rYF17D/SIVGag45-269 during the first 10 days after vaccination with two different doses, as measured by Q-PCR using YF17D-specific primers. FIG. 3B shows the titer of neutralizing antibodies determined at 2 and 5 weeks after vaccination with rYF17D/SIVGag45-269. In FIG. 3C, fresh PBMC from vaccinees (100,00[sic] cells/well) were used in ELISPOT-IFN-γ assays to assess T-cell responses against the YF17D vector and SIV Gag (45-269) insert. YF17D-specific responses were measured using the same epitopes described in FIG. 2. For SIV Gag-specific responses, 6 sets of peptides of 15 mer and with 11 mer overlapping were used, covering the whole extension of Gag 45-269 insert of SIVmac239. In addition, Mamu-A*01-restricted responses against the dominant GagCM9181-189 and subdominant GagQI9254-262 epitopes were measured. The peak of CD8+ T-cell responses in r04091 is depicted at 10 days after the second administration of rYF17D/SIVGag (2.0×10⁵ PFU), which took place on day 28 after the initial vaccination. FIG. 3D shows the expression kinetics of Ki-67 and Bcl-2 in CD8+ T cells after vaccination with rYF17D/SIVGag45-269, as described in FIG. 2.

### Example 4

### Immunization of Rhesus Monkeys with an Initial Dose (Prime) of Recombinant Mycobacterium bovis BCG Followed by a Booster Dose (“boost”) of rYF17D/SIVGag45-269 Virus

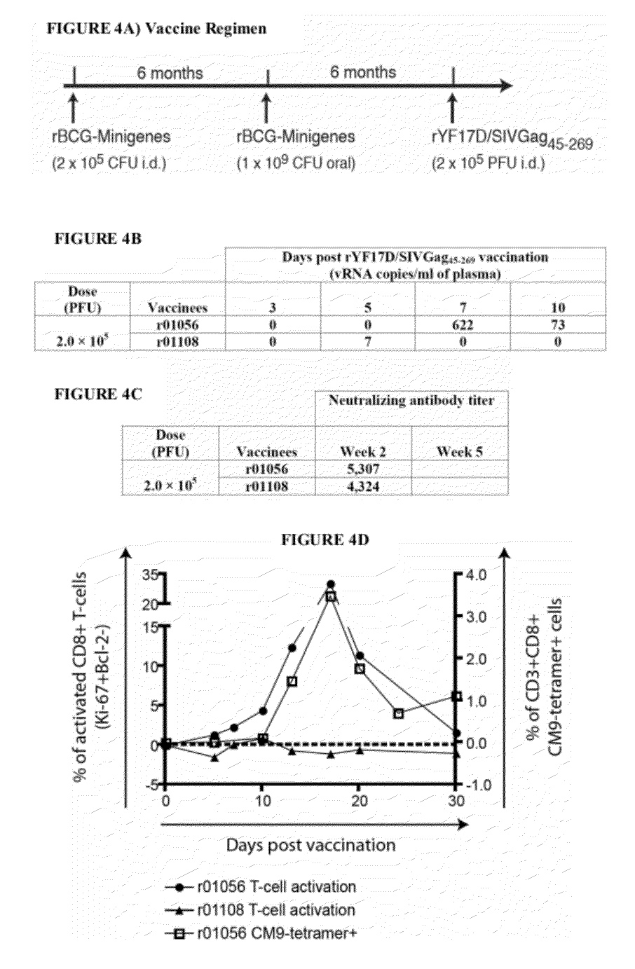

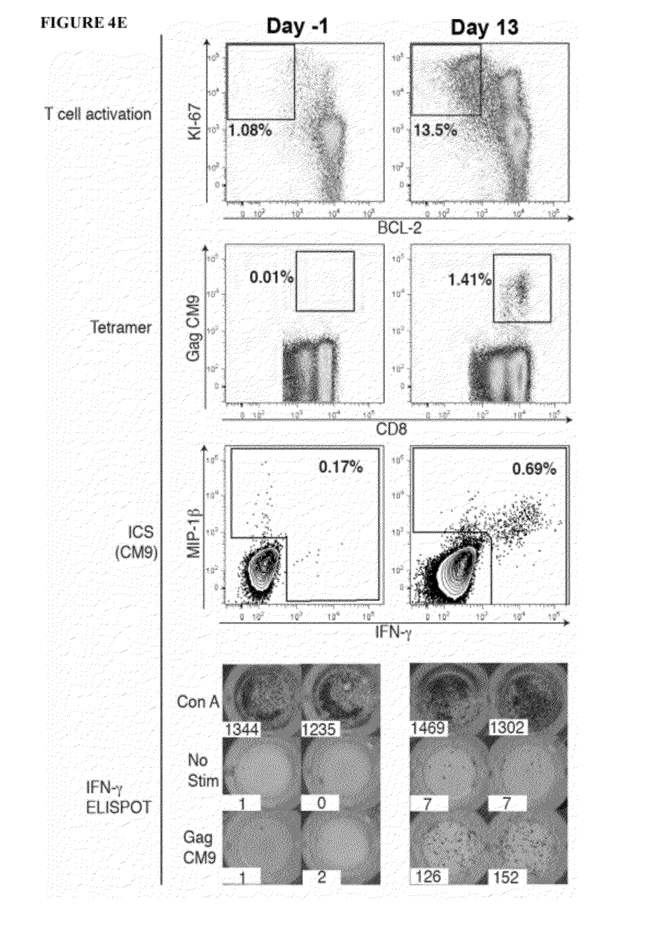

Most viral vectors are usually more efficient after an initial dose [prime] with DNA or rBCG [18, 19, 20, 21]. Thus, two monkeys that had been primed with rBCG expressing SIV proteins (FIG. 4A) were given a booster dose. No SIV-specific response was detected after either of the two priming rBCG vaccinations. Unfortunately, only a low level of rYF17D/SIVGag45-269 replication was observed at day 5 after vaccination in one of the rBCG-primed animals (r01108, 7 copies/mL; FIG. 4B). However, both animals generated neutralizing antibodies at 2 weeks after vaccination (FIG. 4C). Fortunately, high-frequency CD8+ T-cell responses were detected in the Mamu-A*01-positive monkey (r01056) after a booster dose with rYF17D/SIVGag45-269 (FIGS. 4D and 4E). The booster dose induced massive activation of CD8+ T cells in the Mamu-A*01-positive animal r01056, peaking at 35% at 17 days after vaccination (FIGS. 4D and 4E). A frequency of 3.5% of the CD8+ T cells of this animal responded to the Mamu-A*01 GagCM9181-189 epitope at day 17 after vaccination (FIGS. 4D and 4E). These CD8+ T cells synthesized IFN γ, TNFα, MIP-1β and degranulated (FIG. 4E and data not shown).

Thus, an rBCG prime followed by a recombinant yellow fever 17D boost induced polyfunctional antigen-specific CD8+ T cells.

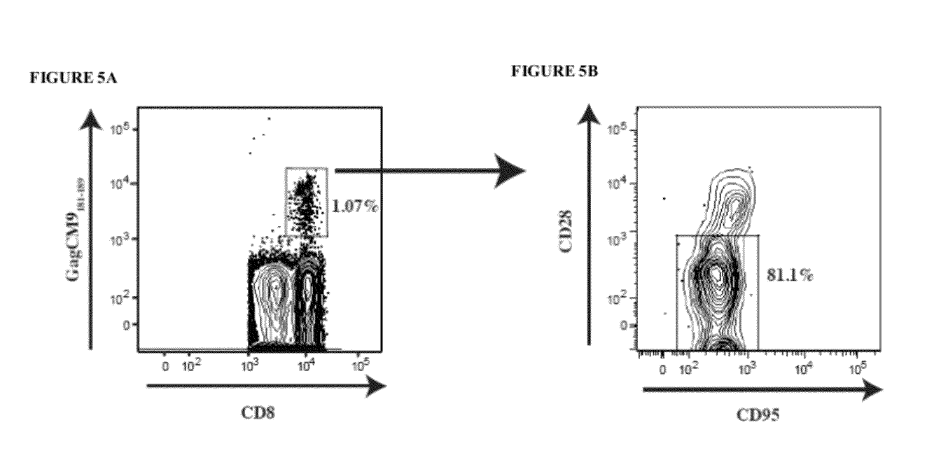

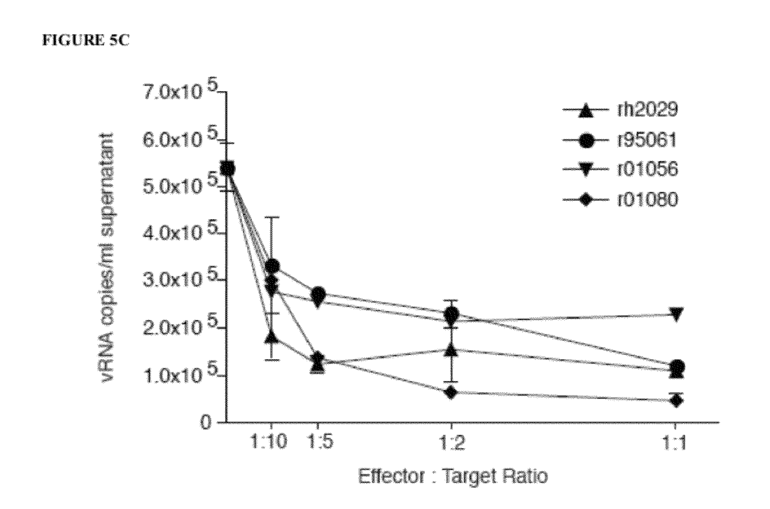

Vaccine-induced CD8+ T cells may be central memory T cells (TCM) or effector memory T cells (TEM). These two subsets of CD8+ T cells differ in function and surface markers [22]. Repeated boosting drives CD8+ T cells toward the TEM subset [22]. Consequently, we evaluated whether an initial dose of rBCG followed by a booster dose of rYF17D/SIVGag45-269 induced TCM or TEM CD8+ T cells. Staining revealed that the SIV-specific CD8+ T cells were largely TEM cells since most of them were CD28+(FIG. 5A). It was recently suggested that TEM cells residing in mucosas can effectively control infection after a low-dose challenge with SIVmac239 [23].

After that, rYF17D/SIVGag45-269-induced CD8+ T cells were tested so as to determine if they could recognize virally infected CD8+ T cells. It was shown that CD8+ T cells stain for tetramers and produce cytokines after stimulation with synthetic peptides. Nevertheless, none of these assays determines whether these CD8+ T cells may recognize SIV-infected cells and reduce viral replication. Thus, a recently developed assay [24] was used to determine whether vaccine-induced CD8+ T cells can reduce viral replication in CD4+ T cells. Sorted tetramer(−) (GagCM9181-189) lymphocytes were incubated for 48 hours with SIVmac239-infected CD4+ T cells that expressed Mamu-A*01. The number of CD4+ T cells that expressed SIV Gag and the quantity of virus in the culture supernatant were assessed. Vaccine-induced CD8+ T cells reduced viral replication to the same extent as that seen with purified SIV-specific CD8+ T cells from three SIVmac239-infected rhesus monkeys, including an elite controller rhesus monkey 95061 (FIG. 5B).

Thus, in one aspect of the methods disclosed here, it is important that the rBCG has induced a high-frequency CD8+ T-cell response after a booster dose of rYF17D/SIVGag45-269. These CD8+ T cells reached frequencies that were similar to those induced by an initial dose of rBCG followed by a booster dose of Ad5 [19]. Even without the benefit of the initial dose of rBCG, the levels of CD8+ T cells induced by a single rYF17D/SIVGag45-269 vaccination were equivalent to those induced by our best SIV vaccine, SIVmac239ANef. Recombinant YF17D generated an average of 195 (range of 100-750) SFC/10 6 [sic] PBMC (N=6), whereas SIVmac239ANef induced an average of 238 (range of 150-320) SFC/10⁶ PBMC (N=3) [25]. It is also possible that any YF17D/HIV recombinants would likely replicate better in humans than in rhesus monkeys, thus inducing more robust immune responses. The rBCG was shown to be more effective in humans [26, 27, 28, 29] and may be more useful for inducing T-cell responses in humans than it has been in our limited study with rhesus monkeys.

FIG. 4 shows that vaccination with rYF17D/SIVGag45-269 induced a robust expansion of Gag-specific responses in an rBCG-primed monkey. FIG. 4A shows the vaccination scheme. Two rhesus monkeys were immunized with rBCG intradermally (i.d.) (2×10⁵ CFU), rBCG orally (10⁹ CFU), and rYF17D/SIVGag45-269 subcutaneously (2×10⁵ PFU) at 6-month intervals. The rBCG was engineered to express 18 minigenes containing sequences of Gag, Vif, Nef, Ver, and Tat from SIVmac239. (See Tables 2-6, Proteins 1(a), 2(a), 3(a), 4(a), 6(a), 8(a), 12(a), 13(a), 14(a), 15(a), 17(a), 18 (a), 19(a), 20 (a), 21 (a), 22 (a), 23(a), e 24(a)). FIG. 4B shows the replication of rYF17D/SIVGag45-269 during the first 10 days after vaccination, as measured by Q-PCR using YF17D-specific primers. FIG. 4C shows the titer of neutralizing antibodies determined at 2 and 5 weeks after vaccination with rYF17D/SIVGag45-269. FIG. 4D shows the kinetics of CD8+ T-cell activation (as described in FIG. 1) and expansion of CM9181-189-specific CD8+ T cells in r01056 after vaccination with rYF17D/SIVGag45-269. In FIG. 4E, it can be seen that vaccination with rYF17D/SIVGag45-269 induces robust CD8+ T-cell responses against GagCM9181-189 in r01056. CD8+ T-cell activation (Ki-67+/Bcl-2-[sic]) for baseline and day 13 are shown. GagCM9181-189-specific responses were measured by tetramer staining, intracellular cytokine staining with antibodies against MIP-1β and IFN-γ, and ELISPOT-IFN-γ.

FIG. 5 illustrates that rYF17D/SIVGag45-269 vaccination of r01056 provides effector memory GagCM9181-189-specific CD8+ T cells that suppresses viral replication in CD4+targets. FIG. 5A shows the frequency of tetramer-positive GagCM9181-189-specific CD8+ T cells in r01056 gated on CD3+CD8+lymphocytes. In FIG. 5B, CD28 and CD95 expression profiles of tetramer-positive cells show a polarized effector memory phenotype. FIG. 5C shows that ex vivo GagCM9181-189-specific CD8+ T cells from r01056 inhibit viral replication from SIVmac239-infected CD4+ T cells. GagCM9181-189-specific CD8+ T cells of three SIV-infected Manu-A*01-positive animals and r01056 vaccinated with rYF17D/SIVGag45-269 were tested for their ability to suppress viral replication from SIV-infected CD4+ T cells [24].

Forty-eight hours after the incubation of various samples containing different ratios of SIV-infected CD4+ T cells and GagCM9191-189-specific CD8+ T cells, the supernatant of these samples was removed and measured for viral RNA copies per milliliter by Q-PCR. No suppression was observed when effectors were incubated with CD4+ targets from Manu-A*01-negative animals. Rh2021 was infected with SIVmac239 (viral load ˜10⁵ vRNA copies/mL) containing mutations in 8 Manu-B*08-restricted epitopes as part of another study [30]. R01080 was vaccinated with a DNA/Ad5 regimen expressing gag, Rev, Tat and Nef, and later infected with SIVmac239 (viral load ˜10³ vRNA copies/mL) [31]. R95061 was vaccinated with a DNA/MVA regimen containing Gag CM9181-189 and was later challenged with SIVmac239 (undetectable viral load) [32].

From the facts exposed above, one may conclude that the attenuated yellow fever vaccine virus 17D expressing SIVmac239 Gag, as described herein, provided excellent immunization responses.

The material above is illustrative of the present invention, and is not intended to limit the scope of the claimed subject matter. The invention is defined by the claims below.

### REFERÊNCIAS

[1]—Burton, D. R., R. C. Desrosiers, R. W. Doms, W. C. Koff, P. D. Kwong, J. P. Moore, G. J. Nabel, J. Sodroski, I. A. Wilson, and R. T. Wyatt, 2004. HIV vaccine design and the neutralizing antibody problem. Nat Immunol 5:233-236.
[2]—Watkins, D. I., D. R. Burton, E. G. Kallas, J. P. Moore, and W. C. Koff. 2008. Nonhuman primate models and the failure of Merck HIV-1 vaccine in humans. Nat med 14:617-621.

[3]—Buchbinder, S. P., D. V. Mehrotra, A. Duerr, D. W. Fitzgerald, R. Mogg, D. Li, P. B. Gilbert, J. R. Lama, M. Marmor, C. Del Rio, M. J. McElrath, D. R. Casimiro, K. M. Gottesdiener, J. A. Chodakewitz, L. Corey, and M. N. Robertson. 2008. Efficacy assessment of a cell-mediated immunity HIV-1 vaccine (the Step Study): a double-blind, randomised, placebo-controlled, test-of-concept trial. Lancet 372:1881-1893.
[4]—McElrath M. J., S. C. De Rosa, Z. Moodie, S. Bubey, L. Kierstead, H. Janes, O. D. Defawe, D. K. Carter, J. Hural, R. Akondy, S. P. Buchbinder, M. N. Robertson, D. V. Mehrotra, S. G. Self, L. Corey, J. W. Shiver, and D. R. Casimiro. 2008. HIV-1 vaccine-induced immunity in the test-of-concept Step Study: a case-cohort analysis. Lancet 372:1894-1905.
[5]—Koup, R. A., J. T. Safrit, Y. Cao, C. A. Andrews, G. McLeod, W. Borkowsky, C. Farthing, and D. D. Ho. Temporal association of cellular immune responses with the initial control of viremia in primary human immunodeficiency virus type 1 syndrome. 1994. J. Virol. 68:4650-4655.
[6]—Friedrich, T. C., L. E. Valentine, L. J. Yant, E. G. Rakasz, S. M. Piaskowski, J. R. Furlott, K. L. Weisgrau, B. Burwitz, G. E. May, E. J. Leon, T. Soma, G. Napoe, S. V. R. Capuano, N. A. Wilson, and D. I. Watkins. 2007. Subdominant CD8+ T-cell responses are involved in durable control of AIDS virus replication. J. Virol. 81:3465-3476.25.
[7]—Loffredo, J. T., E. G. Rakasz, J. P. Giraldo, S. P. Spencer, K. K. Grafton, S. R. Martin, G. Napoé, L. J. Yant, N. A. Wilson, and D. I. Watkins. 2005. Tat28-35SL8-specific CD8+ T lymphocytes are more effective than Gag181-189CM9-specific CD8+ T lymphocytes at suppressing simian immunodeficiency virus replication in a functional in vitro assay. J. Virol. 79: 14986-14991).
[8]—Matano, T., M. Kobayashi, H. Igarashi, A. Takeda, H. Nakamura, M. Kano, C. Sugimoto, K. Mori, A. Iida, T. Hirata, M. Hasegawa, T. Yuasa, M. Miyazawa, Y. Takahashi, M. Yasunami, A. Kimura, D. H. O'Connor, D. I. Watkins, and Y. Nagai. 2004. Cytotoxic T lymphocyte-based control of simian immunodeficiency virus in a preclinical AIDS vaccine trial. J. Exp. Med. 199:1709-1718.
[9]—Poland, J. D., Calisher, C. H., Monath, T. P., Downs, W. G. & Murphy, K. Persistence of neutralizing antibody 30-35 years after immunization with 17D yellow fever vaccine. Bull. World Health Organ. 1981, 59, 895-900;
[10]—Monath, T. P. Yellow fever vaccine. In Vaccines (Ed. Plotkin, S. A. O., W. A.) W.B. Saunders, Philadelphia, 2004. 1095-1176.
[11]—Bontrop R E, Watkins D I. MHC polymorphism: AIDS susceptibility in non-human primates. Trends Immunol. 2005. 26:227-233.
[12]—Valentine L E, Watkins D I. Relevance of studying T cell responses in SIV-infected rhesus macaques. Trends Microbiol. 2008. 16: 605-611.
[13]—Sacha J B, Chung C, Rakasz E G, Spencer S P, Jonas A K, Bean A T, Lee W, Burwitz, B J, Stephany J J, Loffredo J T, Allison D B, Adnan S, Hoji A, Wilson N A, Friedrich, T C, Lifson J D, Yang O O, Watkins D I. 2007. Gag-specific CD8+ T lymphocytes recognize infected cells before AIDS-virus integration and viral protein expression. J. Immunol. 178:2746-2754.
[14]—Peters, B., H. H. Bui, J. Sidney, Z. Weng, J. T. Loffredo, D. I. Watkins, B. R. Mothe, and A. Sette. 2005. A computational resource for the prediction of peptide binding to Indian rhesus macaque MHC class I molecules. Vaccine 23:5212-5224.
[15]—Miller, J. D., R. G. van der Most, R. S. Akondy, J. T. Glidewell, S. Albott, D. Masopust, K. Murali-Krishna, P. L. Mahar, S. Edupuganti, S. Lalor, S. Germon, C. Del Rio, M. J. Mulligan, S. I. Staprans, J. D. Altman, M. B. Feinberg, and R. Ahmed. 2008. Human effector and memory CD8+ T cell responses to smallpox and yellow fever vaccines. Immunity 28:710-722.
[16]—Trindade, G. F., R. S. Marchevsky, A. M. Fillipis, R. M. Nogueira, M. C. Bonaldo, P. C. Acero, E. Caride, M. S. Freire, and R. Galler. 2008. Limited replication of yellow fever 17DD and 17D-Dengue recombinant viruses in rhesus monkeys. An Acad Bras Cienc 80:311-321.
[17]—Wilson, N. A., B. F. Keele, J. S. Reed, S. M. Piaskowski, C. E. MacNair, A. J. Bett, X. Liang, F. Wang, E. Thoryk, G. J. Heidecker, M. P. Citron, L. Huang, J. Lin, S. Vitelli, C. D. Ahn, M. Kaizu, N. J. Maness, M. R. Reynolds, T. C. Friedrich, J. T. Loffredo, E. G. Rakasz, S. Erickson, D. B. Allison, M. J. Piatak, J. D. Lifson, J. W. Shiver, D. R. Casimiro, G. M. Shaw, B. H. Hahn, and D. I. Watkins. 2009. Vaccine-induced cellular heterologous challenge. J Virol 83:6508-6521.
[18]—Allen, T. M., T. U. Vogel, D. H. Fuller, B. R. Mothe, S. Steffen, J. E. Boyson, T. Shipley, J. Fuller, T. Hanke, A. Sette, J. D. Altman, B. Moss, A. J. McMichael, and D. I. Watkins. 2000. Induction of AIDS virus-specific CTL activity in fresh, unstimulated peripheral blood lymphocytes from rhesus macaques vaccinated with a DNA prime/modified vaccinia virus Ankara boost regimen. J Immunol 164:4968-4978.
[19]—Cayabyab, M. J., B. Korioth-Schmitz, Y. Sun, A. Carville, H. Balachandran, A. Miura, K. R. Carlson, A. P. Buzby, B. F. Haynes, W. R. Jacobs, and N. L. Letvin. 2009. Recombinant Mycobacterium bovis BCG prime-recombinant adenovirus boost vaccination in rhesus monkeys elicits robust polyfunctional simian immunodeficiency virus-specific T-cell responses. J Virol 83:5505-5513;
[20]—Hanke, T., R. V. Samuel, T. J. Blanchard, V. C. Neumann, T. M. Allen, J. E. Boyson, S. A. Sharpe, N. Cook, G. L. Smith, D. I. Watkins, M. P. Cranage, and A. J. McMichael. 1999. Effective induction of simian immunodeficiency virus-specific cytotoxic T lymphocytes in macaques by using a multiepitope gene and DNA prime-modified vaccinia virus Ankara boost vaccination regimen. J Virol 73:7524-7532.
[21]—Horton, H., T. U. Vogel, D. K. Carter, K. Vielhuber, D. H. Fuller, T. Shipley, J. T. Fuller, K. J. Kunstman, G. Sutter, D. C. Montefiori, V. Erfle, R. C. Desrosiers, N. Wilson, L. J. Picker, S. M. Wolinsky, C. Wang, D. B. Allison, and D. I. Watkins. 2002. Immunization of rhesus macaques with a DNA prime/modified vaccinia virus Ankara boost regimen induces broad simian immunodeficiency virus (SIV)-specific T-cell responses and reduces initial viral replication but does not prevent disease progression following challenge with pathogenic SIVmac239. J Virol 76:7187-7202.
[22]—Masopust, D., S. J. Ha, V. Vezys, and R. Ahmed. 2006. Stimulation history dictates memory CD8 T cell phenotype: implications for prime-boost vaccination. J Immunol 177:831-839.
[23]—Hansen, S. G., C. Vieville, N. Whizin, L. Coyne-Johnson, D. C. Siess, D. D. Drummond, A. W. Legasse, M. K. Axthelm, K. Oswald, C. M. Trubey, M. J. Piatak, J. D. Lifson, J. A. Nelson, M. A. Jarvis, and L. J. Picker. 2009. Effector memory T cell responses are associated with protection of rhesus monkeys from mucosal simian immunodeficiency virus challenge. Nat Med 15:293-299.
[24]—Vojnov, L. J. Reed, K. L. Weisgrau, E. Rakasz, J. T. Loffredo, S. Piaskowski, J. B. Sacha, H. L. Kolar, N. A. Wilson, R. P. Johnson, and D. I. Watkins. Effective SIV-specific CD8+ T-cells lack an easily detectable, shared characteristic. J Virol Manuscript in press.
[25]—Reynolds, M R, A. M. Weiler, K. L. Weisgrau, S. M. Piaskowski, J. R. Furlott, J. T. Weinfurther, M Kaizu, T. Soma, E. J. Leon, C. MacNair, D. P. Leaman, M. B. Zwick, E. Gostick, S. K. Musani, D. A. Price, T. C. Friedrich, E. G. Rakasz, N. A. Wilson, A. B. McDermott, R. Boyle, D. B. Allison, D. R. Burton, W. C. Koff, and D. I. Watkins. 2008. Macaques vaccinated with live-attenuated SIV control replication of heterologous virus. J Exp Med 205:2537-2550.
[26]—Barker, L. F., M. J. Brennan, P. K. Rosenstein, and J. C. Sadoff. 2009. Tuberculosis vaccine research: the impact of immunology. Curr Opin Immunol 21:331-338.
[27]—Hoft, D. F., A. Blazevic, G. Abate, W. A. Hanekom, G. Kaplan, J. H. Soler, F. Weichold, L. Geiter, J. C. Sadoff, and M. A. Horwitz. 2008. A new recombinant bacille Calmette-Guerin vaccine safely induces significantly enhanced tuberculosis-specific immunity in human volunteers. J Infect Dis 198:1491-1501.
[28]—Skeiky, Y. A., and J. C. Sadoff. 2006. Advances in tuberculosis vaccine strategies. Nat Rev Microbiol 4:469-476
[29]—Stover, C. K., G. P. Bansal, S. Langerman, and M. S. Hanson. 1994. Protective immunity elicited by rBCG vaccines. Dev Biol Stand 82:163-170.
[30]—Valentine, L. E., J. T. Loffredo, A. T. Bean, E. J. Leon, C. E. Macnair, D. R. Beal, S. M. Piaskowski, Y. C. Klimentidis, S. M. Lank, R. W. Wiseman, J. T. Weinfurter, G. E. May, E. G. Rakasz, N. A. Wilson, T. C. Friedrich, D. H. O'Connor, D. B. Allison, and D. I. Watkins. 2009. Infection with “escaped” virus variants impairs control of SIVmac239 replication in Mamu-B*08+macaques. J. Virol., in press.
[31]—Wilson, N. A., J. Reed, G. S, Napoe, S. Piaskowski, A. Szymanski, J. Furlott, E. J Gonzalez, L. J. Yant, N. J. Maness, G. E. May, T. Soma, M. R. Reynolds, E. Rakasz, R. Rudersdorf, A. B. MacDermott, D. H. O'Connor, T. C. Friedrich, D. B. Alison, A. Patki, L. J. Picker, D. R. Burton, J. Lin, L. Huang, D. Patel, G. Heindecker, J. Fan, M. Citron, M. Horton, F. Wang, X. Liang, J. W. Shiver, D. R. Casimiro, and D. I. Watkins. 2006. Vaccine-induced cellular immune responses reduce plasma viral concentrations after repeated low-dose challenge with pathogenic simian immunodeficiency virus SIVmac239. J Virol 80:5875-5885.
[32]—Allen, T. M., P. Jing, B. Calore, H. Horton, D. H. O'Connor, T. Hanke, M. Piekarczyk, R. Ruddersdorf, B. R. Mothe, C. Emerson, N. Wilson, J. D. Lifson, I. M. Belyakov, J. A. Berzofsky, C. Wang, D. B. Allison, D. C. Montefiori, R. C. Desrosiers, S. Wolinsky, K. J. Kunstman, J. D. Altman, A. Sette, A. J. McMichael, and D. I. Watkins. 2002. Effects of cytotoxic T lymphocytes (CTL) directed against a single simian immunodeficiency virus (SIV) Gag CTL epitope on the course of SIVmac239 infection. J Virol 76:10507-10511.

1. A pharmaceutical composition for infection caused by lentiviruses, comprising:
(A) a recombinant yellow fever virus which expresses a heterologous polypeptide comprising at least a fragment of a lentiviral polypeptide, and
(B) a pharmaceutical carrier;
wherein the composition comprises the recombinant yellow fever virus in an amount effective to induce a therapeutic or protective immune response against infection with lentivirus.

2. The composition according to claim 1, wherein the heterologous polypeptide includes at least a fragment of an HIV Gag polypeptide or at least a fragment of athe polypeptide of SIV Gag.

3. The composition according to claim 1, wherein the heterologous polypeptide includes at least a fragment of SEQ ID NO:9.

4. The composition according to claim 1, wherein the fragment comprises at least 8, 9, 10, 20, 30, 40, 50, 100 or 200 continuous amino acids continuous of SEQ ID NO:9.

5. The composition according to claim 1, wherein the fragment comprises amino acids 45-269 of SEQ ID NO:9.

6. The composition according to claim 1, wherein the heterologous polypeptide comprises a sequence of amino acid C-terminal comprising KESSIG.

7. The composition according to claim 1, wherein the recombinant yellow fever virus is YF17D.

8. The composition according to claim 1, wherein the recombinant yellow fever virus comprises a sequence coding for the heterologous polypeptide inserted between sequences coding for polypeptide and NS1 polypeptide.

9. The composition according to claim 1 characterized in that the recombinant yellow fever virus comprises a sequence coding for the heterologous polypeptide, said sequence being optimized according to codon usage frequency of the yellow fever virus.

10. A pharmaceutical composition for infection caused by lentiviruses, comprising a mixture of recombinant yellow fever viruses, wherein the mixture comprises:
(A) a first recombinant yellow fever virus which expresses a first heterologous polypeptide comprising at least a fragment of a lentiviral Gag polypeptide;
(B) a second recombinant yellow fever virus which expresses a second heterologous polypeptide comprising at least a fragment of a lentiviral Vif polypeptide;
(C) a third recombinant yellow fever virus which expresses a third heterologous polypeptide comprising at least a fragment of a lentiviral Nef polypeptide, and
(D) a pharmaceutical carrier,
wherein the composition comprises the mixture in an amount effective to induce a protective or therapeutic immune response against infection lentiviral.

11. The composition according to claim 10, wherein the Gag polypeptide is a Gag polypeptide of HIV; the Vif polypeptide is a Vif polypeptide of HIV; and the Nef polypeptide is a Nef polypeptide of HIV.

12. A method for inducing a protective or therapeutic immune response against HIV, wherein the method comprises administering the composition defined in claim 1 to an individual who needs it.

13. The method according to claim 12, further comprising administering an initial dose of the composition of claim 1 in a priming step prior to administering the composition of claim 1, wherein the initial dose comprises a DNA encoding one or more of HIV polypeptides or fragments thereof and/or the initial dosage of the composition comprises rBCG that expresses one or more of polypeptides of HIV or fragments thereof.

14. The method according to claim 12, wherein the composition is administered in two or more subsequent times, waiting for at least 4-12 weeks before the subsequent administration.

15. The method according to claim 12, wherein a protective or therapeutic immune response against HIV includes a response of CD4+ T cell and/or a response in CD8+ Tcell.

16. A vaccine to induce a protective immune response or a therapeutic response against HIV, wherein said vaccine comprises:
(A) a recombinant yellow fever virus which expresses a heterologous polypeptide comprising at least a fragment of a lentiviral polypeptide;
(B) a pharmaceutical carrier;
wherein the composition comprises the recombinant yellow fever virus in an amount effective to induce a therapeutic or protective immune response against infection with lentivirus.

17. A vaccine to induce a protective immune response or therapeutic response against HIV, wherein said vaccine comprises a mixture of recombinant yellow fever virus, said mixture comprising:
(A) a first recombinant yellow fever virus which expresses a first heterologous polypeptide comprising at least a fragment of a lentiviral Gag polypeptide;
(B) a second recombinant yellow fever virus expressing a second heterologous polypeptide comprising at least a fragment of a lentiviral Vif polypeptide;
(C) a third recombinant yellow fever virus that expresses a third heterologous polypeptide comprising at least a fragment of a lentiviral Nef polypeptide; and
(D) a pharmaceutical carrier.

18. A 45-269 amino acid fragment of the Gag protein used for drawing a heterologous cassette of SIV, wherein the fragment comprises SEQ ID NO:1.

19. A recombinant protein of 45-269 amino acids from Gag protein, wherein the recombinant protein comprises SEQ ID NO:2.

20. A recombinant gene of Gag 45-269 protein of SIV, wherein the gene comprises SEQ ID NO:6.

21. A nucleotide sequence of recombinant yellow fever virus which expresses Gag 45-269 protein, wherein the nucleotide sequence comprises SEQ ID NO:7.

22. A precursor polyprotein containing a recombinant protein of amino acids 45-269 from the SIV Gag protein, wherein the precursor polyprotein comprises SEQ ID NO:8.

23. A method for producing lentivirus proteins or related polypeptides, wherein said method comprises the steps of:
(A) introducing at least one vector or a vector system in a host cell, said vector or vector system including a nucleic acid sequence encoding at least an immunogenic protein of lentivirus or a related polypeptide, a nucleic acid sequence having at least 90% identity with sequence SEQ ID NO:6 or SEQ ID NO:7 or an encoded protein having at least 90% identity with a protein defined by SEQ ID NOs:1, 2, 8 or 9;
(B) expressing the nucleic acid sequence into the host cell; and
(C) producing anthe antigenic protein encoded by lentivirus or a related polypeptide within the host cell.

24. The method according to claim 23, wherein two or more vectors or vector systems are introduced.

25. The method according to claim 23, wherein the vector or vector system includes a nucleic acid sequence encoding two or more immunogenic proteins of lentiviruses or related polypeptides.
